SEQUENCE LISTING (29 sequences)

SEQ ID NO: 1 - protein, 5 aa, synthetic construct
  REGION 1..5: note = Description of Artificial Sequence: Synthetic peptide
SFGMH

SEQ ID NO: 2 - protein, 17 aa, synthetic construct
  REGION 1..17: note = Description of Artificial Sequence: Synthetic peptide
YISSGSSTIYYGDTVKG

SEQ ID NO: 3 - protein, 15 aa, synthetic construct
  REGION 1..15: note = Description of Artificial Sequence: Synthetic peptide
EGGYYYGRSYYTMDY

SEQ ID NO: 4 - protein, 16 aa, synthetic construct
  REGION 1..16: note = Description of Artificial Sequence: Synthetic peptide
RSSQSIVHSNGNTYLE

SEQ ID NO: 5 - protein, 7 aa, synthetic construct
  REGION 1..7: note = Description of Artificial Sequence: Synthetic peptide
KVSNRFS

SEQ ID NO: 6 - protein, 9 aa, synthetic construct
  REGION 1..9: note = Description of Artificial Sequence: Synthetic peptide
FQGSHVPPT

SEQ ID NO: 7 - protein, 5 aa, synthetic construct
  REGION 1..5: note = Description of Artificial Sequence: Synthetic peptide
TYWIT

SEQ ID NO: 8 - protein, 17 aa, synthetic construct
  REGION 1..17: note = Description of Artificial Sequence: Synthetic peptide
DIYPGSSISNYNEKFKS

SEQ ID NO: 9 - protein, 10 aa, synthetic construct
  REGION 1..10: note = Description of Artificial Sequence: Synthetic peptide
EDGYDAWFAY

SEQ ID NO: 10 - protein, 16 aa, synthetic construct
  REGION 1..16: note = Description of Artificial Sequence: Synthetic peptide
RSSQSILHSNGNTYLE

SEQ ID NO: 11 - protein, 7 aa, synthetic construct
  REGION 1..7: note = Description of Artificial Sequence: Synthetic peptide
KVSNRFS

SEQ ID NO: 12 - protein, 9 aa, synthetic construct
  REGION 1..9: note = Description of Artificial Sequence: Synthetic peptide
FQGSHVPFT

SEQ ID NO: 13 - protein, 124 aa, synthetic construct
  REGION 1..124: note = Description of Artificial Sequence: Synthetic polypeptide
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 14 - protein, 112 aa, synthetic construct
  REGION 1..112: note = Description of Artificial Sequence: Synthetic polypeptide
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 15 - protein, 119 aa, synthetic construct
  REGION 1..119: note = Description of Artificial Sequence: Synthetic polypeptide
EVQLLESGGGLVQPGGSLRLSCAASGYTFTTYWITWVRQAPGKGLEWVSDIYPGSSISNYNEKFKSRFTISVDNSKNTLYLQMNSLRAEDTAVYYCAREDGYDAWFAYWGQGTLVTVSS

SEQ ID NO: 16 - protein, 112 aa, synthetic construct
  REGION 1..112: note = Description of Artificial Sequence: Synthetic polypeptide
DIQMTQSPSSLSASVGDRVTITCRSSQSILHSNGNTYLEWYQQKPGKAPKLLISKVSNRFSGVPSRFSGSGSGTDFTLTISSLQPEDFATYYCFQGSHVPFTFGQGTKLEIK

SEQ ID NO: 17 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 107 aa, synthetic construct [sequence omitted]

SEQ ID NO: 19 - protein, 330 aa, synthetic construct [sequence omitted]

SEQ ID NO: 20 - protein, 107 aa, synthetic construct [sequence omitted]

SEQ ID NO: 21 - protein, 40 aa, Homo sapiens
DAEFRHDSGYEVHHQKLVFFAEDVGSNKGAIIGLMVGGVV

SEQ ID NO: 22 - protein, 42 aa, Homo sapiens [sequence omitted]

SEQ ID NO: 23 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 24 - protein, 8 aa, synthetic construct
  REGION 1..8: note = Description of Artificial Sequence: Synthetic peptide
GYTFTTYW

SEQ ID NO: 25 - protein, 8 aa, synthetic construct
  REGION 1..8: note = Description of Artificial Sequence: Synthetic peptide
IYPGSSIS

SEQ ID NO: 26 - protein, 12 aa, synthetic construct
  REGION 1..12: note = Description of Artificial Sequence: Synthetic peptide
AREDGYDAWFAY

SEQ ID NO: 27 - protein, 11 aa, synthetic construct
  REGION 1..11: note = Description of Artificial Sequence: Synthetic peptide
QSILHSNGNTY

SEQ ID NO: 28 - skipped

SEQ ID NO: 29 - protein, 9 aa, synthetic construct
  REGION 1..9: note = Description of Artificial Sequence: Synthetic peptide
FQGSHVPFT

(19) United States
(12) Patent Application Publication
(10) Pub. No.: US 2025/0051427 A1
(43) Pub. Date: Feb. 13, 2025

(54) METHODS OF USING AN ANTI-AMYLOID BETA PROTOFIBRIL ANTIBODY AND ANTI-TAU ANTIBODY

(71) Applicants: EISAI R&D MANAGEMENT CO., LTD., Tokyo (JP); WASHINGTON UNIVERSITY, St.Louis, MO (US)

(72) Inventors: Larisa REYDERMAN, Nutley, NJ (US); Jin ZHOU, Nutley, NJ (US); Lynn KRAMER, Nutley, NJ (US); Michael IRIZARRY, Nutley, NJ (US); Pallavi SACHDEV, Nutley, NJ (US); Shobha DHADDA, Nutley, NJ (US); David LI, Nutley, NJ (US); Kristin WILDSMITH, Nutley, NJ (US); Pau ACEVES BALDO, Hatfield Hertfordshire (GB); Sumit RAWAL, Nutley, NJ (US); Akihiko KOYAMA, Nutley, NJ (US); Chad SWANSON, Nutley, NJ (US); Michio KANEKIYO, Nutley, NJ (US); June KAPLOW, Nutley, NJ (US); David VERBEL, Nutley, NJ (US); Ishani LANDRY, Nutley, NJ (US); Seiichi HAYATO, Tokyo (JP); Robert GORDON, Hatfield Hertfordshire (GB); Randall BATEMAN, St. Louis, MO (US); Eric MCDADE, St.Louis, MO (US)

(21) Appl. No.: 18/720,518

(22) PCT Filed: Nov. 8, 2022
(86) PCT No.: PCT/US2022/079509

§ 371 Date: Jun. 14, 2024

Related U.S. Application Data
(60) Provisional application No. 63/291,315, filed on Dec. 17, 2021.

(30) Foreign Application Priority Data
Dec. 17, 2021 (WO) PCT/IB2021/000937

(51) Int. Cl. C07K 16/18; A61K 39/00

(52) U.S. Cl. CPC C07K 16/18; A61K 2039/545

(57) ABSTRACT

Disclosed herein are antibodies, pharmaceutical formulations for treating or preventing Alzheimer's disease, methods of treating or preventing Alzheimer's disease, and kits comprising pharmaceutical formulations for treating or preventing Alzheimer's Disease comprising an anti-Aβ protofibril antibody and anti-tau antibody.

This application claims the benefit of priority of U.S. Provisional Application No. 63/291,315, filed Dec. 17, 2021, and International Application No. PCT/IB2021/000937, filed Dec. 17, 2021, the contents of which are incorporated herein by reference in their entirety.

This invention was made with government support under grant numbers U01AG042791, R01AG046179, R56AG053267, U01AG059798, R01AG053267, and R01AG068319, awarded by the National Institute of Health. The Government has certain rights in the invention.

The instant application contains a Sequence Listing which has been submitted electronically in ASCII format and is hereby incorporated by reference in its entirety. Said ASCII copy, created on Dec. 3, 2021, is named 08061_0053-00304_SL.txt and is 21,812 bytes in size.

Alzheimer's disease (AD) is a progressive, neurodegenerative disorder and the most common form of dementia among older people. In 2006, there were 26.6 million cases of AD in the world (range: 11.4-59.4 million) (Brookmeyer, R., et al., Forecasting the global burden of Alzheimer's Disease. Alzheimer Dement. 2007; 3:186-91), while there were more than 5 million people in the United States reportedly living with AD (2010 Alzheimer's disease facts and figures. Alzheimer Dement. 2010; 6:158-94). By the year 2050, the worldwide prevalence of AD is predicted to grow to 106.8 million (range: 47.2-221.2 million), while in the United States alone the prevalence is estimated to be 11 to 16 million. (Brookmeyer, supra, and 2010 Alzheimer's disease facts and figures, supra).

The disease generally involves a global decline of cognitive function that progresses slowly and leaves end-stage subjects bedridden. AD subjects typically survive for only 3 to 10 years after symptom onset, although extremes of 2 and 20 years are known. (Hebert, L. E., et al., Alzheimer disease in the U.S. population: prevalence estimates using the 2000 census. Arch Neurol. 2003; 60:1119-1122.) AD is the seventh leading cause of all deaths in the United States and the fifth leading cause of death in Americans older than the age of 65 years, despite the fact that mortality due to AD is greatly underestimated because death certificates rarely attribute the cause of death to AD. (2010 Alzheimer's disease facts and figures, supra.)

Histologically, the disease is characterized by neuritic plaques, found primarily in the association cortex, limbic system and basal ganglia. The major constituent of these plaques is amyloid beta peptide (Aβ). Aβ exists in various conformational states—monomers, oligomers, protofibrils, and insoluble fibrils.

In addition to Aβ plaques, Alzheimer's disease is also characterized by the presence of accumulation of Tau-containing neurofibrillary tangles. Human Tau is encoded by the microtubule-associated protein Tau gene, MAPT, located on chromosome 17q21. The adult human brain contains six main Tau isoforms which are generated by alternative splicing of exon 2 (E2), E3, and E10. These isoforms differ depending on the number of 29-residue repeat regions near the N-terminus. Tau isoforms containing 0, 1, or 2 inserts are known as 0N, 1N, and 2N, respectively. Unprocessed Tau isoforms also contain either 3 (“3R”) or 4 (“4R”) microtubule-binding repeat domains. The second of these repeat domains is encoded by E10 and is not included in 3R Tau isoforms.

Although Tau is usually highly soluble, under pathological conditions, it can aggregate into paired helical filaments, neurofibrillary tangles and other structures that define a large spectrum of neurodegenerative diseases termed Tauopathies. Tauopathy thus refers to a class of neurodegenerative diseases associated with aggregation of the microtubule-associated protein Tau, including Alzheimer's disease (AD), progressive supranuclear palsy (PSP), and frontotemporal dementia (FTD).

Details of the mechanistic relationship between onset of Alzheimer's disease, Aβ production, and Tau-mediated neurotoxicity is poorly understood. There remains a potential for improved therapies that target both Aβ production and Tau-containing neurofibrillary tangles. Thus, there is a need for specific and effective therapeutic agents that target Aβ and Tau.

Provided herein are methods for treating and/or preventing Alzheimer's disease comprising administering to a subject in need thereof an anti-Aβ protofibril antibody or antigen binding fragment thereof that is capable of binding to a human Aβ protofibril and an anti-tau antibody or antigen binding fragment thereof that is capable of binding to human tau.

In some embodiments, the method of treating or preventing Alzheimer's disease in a subject in need thereof comprises administering to the subject:
(i) an isolated anti-Aβ protofibril antibody or antigen binding fragment thereof that is capable of binding to a human Aβ protofibril comprising
   (a) a heavy chain variable domain comprising an amino acid sequence of SEQ ID NO:13, and
   (b) a light chain variable domain comprising an amino acid sequence of SEQ ID NO:14, and
(ii) an anti-tau antibody or antigen binding fragment thereof that is capable of binding to human tau comprising
   (a) a heavy chain variable domain comprising an amino acid sequence of SEQ ID NO:15, and
   (b) a light chain variable domain comprising an amino acid sequence of SEQ ID NO:16, wherein the anti-Aβ protofibril antibody or fragment thereof is administered in conjunction (e.g., simultaneously or sequentially) with the anti-tau antibody or fragment thereof. In some embodiments, the isolated anti-Aβ protofibril antibody or fragment thereof is administered once every two weeks. In some embodiments, the anti-tau antibody or fragment thereof is administered once every four weeks. In some embodiments, the anti-Aβ protofibril antibody or fragment thereof is administered before the start of treatment with the anti-tau antibody or fragment thereof, e.g., when, the subject is symptomatic for Alzheimer's disease. In some embodiments, the anti-tau antibody or fragment thereof is administered before the start of treatment with the anti-Aβ protofibril antibody or fragment thereof, e.g., when the subject is asymptomatic for Alzheimer's disease. In some embodiments, the anti-Aβ protofibril antibody or fragment thereof is administered at a dose of 5 mg/kg-20 mg/kg, e.g., wherein the dose is 10 mg/kg. In some embodiments, the anti-tau antibody or fragment thereof is administered in an amount of 1000-45000 mg, e.g., wherein the dose is 1500 mg. In some embodiments, the subject has a genetic mutation for dominantly inherited Alzheimer's disease, e.g., wherein the subject a genetic mutation in at least one of three genes—PSEN1, PSEN2, or APP.

Also provided herein are kits and pharmaceutical combinations comprising
(i) an isolated anti-Aβ protofibril antibody or antigen binding fragment thereof that is capable of binding to a human Aβ protofibril comprising
   (a) a heavy chain variable domain comprising an amino acid sequence of SEQ ID NO:13, and
   (b) a light chain variable domain comprising an amino acid sequence of SEQ ID NO:14, and
(ii) an anti-tau antibody or antigen binding fragment thereof that is capable of binding to human tau comprising
   (a) a heavy chain variable domain comprising an amino acid sequence of SEQ ID NO:15, and
   (b) a light chain variable domain comprising an amino acid sequence of SEQ ID NO:16,
wherein the anti-Aβ protofibril antibody or fragment thereof is administered in conjunction (e.g., simultaneously or sequentially) with the anti-tau antibody or fragment thereof.

### BRIEF DESCRIPTION OF THE DRAWINGS

FIG. 1 depicts the randomization scheme for treatment with E2814 concurrent with lecanemab.

FIG. 2 depicts the E2814 or placebo and open-label lecanemab treatment schemes.

### DEFINITIONS

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B,” when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When a number is recited, either alone or as part of a numerical range, it should be understood that the numerical value can vary above and below the stated value by a variance of 10% of the stated value.

The term “antibody” as used herein is meant in a broad sense and includes immunoglobulin or antibody molecules including polyclonal antibodies, monoclonal antibodies including murine, human, human-adapted, humanized and chimeric monoclonal antibodies and antibody fragments. In general, antibodies are proteins or peptide chains that exhibit binding specificity to a specific antigen. Intact antibodies are typically heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (variable region) (VH) followed by a number of constant domains (constant regions). Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains.

Immunoglobulins can be assigned to five major classes or isotypes, depending upon the type of constant domain possessed by its heavy chain, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. The heavy chain constant domains that correspond to the different classes of immunoglobulins are called α, δ, ε, γ, and μ, respectively.

An immunoglobulin light chain variable region or heavy chain variable region consists of a “framework” region interrupted by three Complementarity Determining Regions (CDRs) that provide the main determinants of antigen binding (Wu and Kabat, J. Exp. Med. 132:211-250, 1970). Generally, the antigen-binding site has six CDRs; three in the VH (HCDR1, HCDR2, HCDR3), and three in the VL (LCDR1, LCDR2, LCDR3) (Kabat et al., Sequences of Proteins of Immunological Interest, 5th Ed. Public Health Service, National Institutes of Health, Bethesda, Md., 1991). The CDRs may be determined according to the Kabat numbering scheme. SEQUENCES OF PROTEINS OF IMMUNOLOGICAL INTEREST (Kabat et al., 5th ed., U.S. Department of Health and Human Services, NIH Publication No. 91-3242, 1991, hereafter referred to as “Kabat report”). Alternatively, the “IMGT-CDRs” as proposed by Lefranc (Lefranc et al., Dev. Comparat. Immunol. 27:55-77, 2003) are based on the comparison of V domains from immunoglobulins and T-cell receptors. The International ImMunoGeneTics (IMGT) database (http://www_imgt_org) provides a standardized numbering and definition of these regions. The correspondence between CDRs and IMGT delineations is described in Lefranc et al., Dev. Comparat. Immunol. 27:55-77, 2003.

Antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “antibody or antigen-binding fragment thereof” may be used to denote that a given antigen-binding fragment incorporates one or more amino acid segments of the antibody referred to in the phrase.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

In some embodiments, the subject has “elevated amyloid” or “intermediate amyloid.” In some embodiments, the level of amyloid is measured using amyloid PET. As one of ordinary skill in the art will recognize, amyloid levels from amyloid PET can be reported using the Centiloid method in “centiloid” units (CL). (Klunk W E et al. The Centiloid Project: standardizing quantitative amyloid plaque estimation by PET. Alzheimer 's Dement. 2015; 11:1-15 el-4). The Centiloid method measures a tracer on a scale of 0 CL to 100 CL, where 0 is deemed the anchor-point and represents the mean in young healthy controls and 100 CL represents the mean amyloid burden present in subjects with mild to moderate severity dementia due to AD. (Id.) As is known to one of ordinary skill in the art, centiloid thresholds may vary, for example may be refined, based on new or additional scientific information. (See, e.g., http://www.gaain.org/centiloid-project.) An elevated level of amyloid can be set relative to a baseline threshold in a healthy control determined according to methods known to a POSA. For example, a centiloid value of 32.5 can be used as a threshold value for “elevated amyloid,” and an “intermediate amyloid” level can refer to an Aβ amyloid PET in the range of 20-32.5 CL. In another example, a centiloid value of 40 can be used as a threshold value for “elevated amyloid,” and an “intermediate amyloid” level can refer to an Aβ amyloid PET in the range of 20-40 CL. Rowe et al., Eur J Nucl Med Mol I 44, 2053-2059 (2017); Salvad6 et al., Alzheimer's Res Ther 11, 27 (2019); Sabri et al., Alzheimers Dement 11: 964-74 (2015); Rowe et al., Alzheimer's Dementia 14, P634 (2018); Amadoru et al., Alzheimer's Res Ther 12, 22 (2020); Roé-Vellvé et al., Alzheimer's Dementia 16, (2020); Bullich et al., Alzheimer's Res Ther 13, 67 (2021).

Subjects that are “asymptomatic for Alzheimer's disease,” as described herein, are cognitively normal subjects with intermediate or elevated levels of amyloid in the brain and can be identified by asymptomatic stages (e.g., based on Aβ accumulation in the brain and/or by CSF or blood based biomarkers) with or without memory complaints and emerging episodic memory and executive function deficits. Cognitively normal can include subjects who are CDR 0, or subjects within the normal ranges of cognitive test scores. Asymptomatic AD occurs prior to significant irreversible neurodegeneration and cognitive impairment and is typically characterized by the appearance of in vivo molecular biomarkers of AD and the absence clinical symptoms. Asymptomatic AD biomarkers that may suggest the development of Alzheimer's disease include, but are not limited to, one or more of intermediate or elevated levels of amyloid and/or tau in the brain, e.g., as measured by amyloid or tau positron emission tomography (PET), cerebrospinal fluid level of Aβ1-42, cerebrospinal fluid level of total tau, cerebrospinal fluid level of neurogranin, cerebrospinal fluid level of neurofilament light chain, and blood biomarkers as measured in the serum or plasma (e.g. levels of Aβ1-42, the ratio of two forms of amyloid-β peptide (Aβ42/Aβ40), plasma levels of total tau (T-tau), levels of phosphorylated tau (P-tau) isoforms (including tau phosphorylated at 181 (P-tau181) 217 (P-tau217), and 231 (P-tau231)) and neurofilament light (NfL)). For example, it has been found that subjects treated with elenbecestat (E2609), a β-site amyloid precursor protein cleaving enzyme (BACE) inhibitor, who had amyloid baseline positron emission tomography (PET) standard uptake value ratios (SUVr values) of 1.4 to 1.9, exhibited the greatest slowing of cognitive decline while on treatment. See Lynch, S. Y. et al. “Elenbecestat, a BACE inhibitor: results from a Phase 2 study in subjects with mild cognitive impairment and mild-to-moderate dementia due to Alzheimer's disease.” Poster P4-389, Alzheimer's Association International Conference, Jul. 22-26, 2018, Chicago, IL, USA. Similarly, it has been found that subjects having a baseline florbetapir amyloid PET SUVr levels below 1.2 do not exhibit enough cognitive decline to be detectable, whereas subjects having SUVr levels above 1.6 appear to correlate with a plateau effect in which amyloid level has reached a saturation level and treatment does not result in a change of cognitive measures. See Dhadda, S. et al., “Baseline florbetapir amyloid PET standard update value ratio (SUVr) can predict clinical progression in prodromal Alzheimer's disease (pAD).” Poster P4-291, Alzheimer's Association International Conference, Jul. 22-26, 2018, Chicago, IL, USA; Sperling R A et al., Alzheimer's Dement. 2011; 7:280-92; Jack C. R. et al., Alzheimer's Dement. 2018; 14:535-62.

“Early-onset Alzheimer's disease,” as used herein, refers to a continuum of AD severity from mild cognitive impairment due to AD—intermediate likelihood to mild Alzheimer's disease dementia. Subjects with early-onset AD include subjects with mild Alzheimer's disease dementia as defined herein and subject with mild cognitive impairment (MCI) due to AD—intermediate likelihood as defined herein. In some embodiments, subjects with early-onset AD have a score of 22-30 on the Mini-Mental State Examination (MMSE) and/or a CDR global range 0.5 to 1.0.

Subjects with “mild Alzheimer's disease dementia,” or “mild AD dementia” as used herein, are subjects meeting the National Institute of Aging-Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 2011; 7:263-9. Also included herein are subjects who have a CDR score of 0.5 to 1.0 and/or a Memory Box score of 0.5 or greater at screening and baseline.

Subjects with “mild cognitive impairment due to AD—intermediate likelihood,” as used herein are those identified as such in accordance with the NIA-AA core clinical criteria for mild cognitive impairment due to Alzheimer's disease—intermediate likelihood (see McKhann supra). For example, a subject may be symptomatic but not demented, with evidence of brain amyloid pathology making them less heterogeneous and more similar to mild Alzheimer's disease dementia subjects in cognitive and functional decline as measured by CDR with a score of 0.5 and/or a Memory Box score of 0.5 or greater at screening and baseline. Furthermore, subjects who report a history of subjective memory decline with gradual onset and slow progression over the last 1 year before screening, and which is corroborated by an informant, are also included herein.

A subject's amyloid level can be detected by biomarkers such as, but not limited to: (a) amyloid detected by PET scan from either a visual read or semiquantitative thresholds (SUVr or centiloid); (c) cerebrospinal fluid (CSF) Aβ1-42, and/or Aβ1-42/1-40 ratio; and/or (d) blood biomarkers (i.e. plasma Aβ1-42, Aβ1-42/Aβ1-40, tau, total tau (T-tau), P-tau, and/or NfL). Secondary markers may confirm a primary amyloid determination and include, but are limited to: (a) tau detected by a PET scan; (b) CSF tau, phosphorylated tau (p-tau), neurofilament light peptide (NfL), and/or neurogranin; (c) other blood biomarkers (i.e. tau, total tau (T-tau), P-tau, and/or NfL).

“Amyloid” refers to fibers that are unbranched, usually extracellular, and found in vivo; in addition, the fibers bind the dye Congo Red and then show green birefringence when viewed between crossed polarizers. Amyloid-forming proteins have been identified and associated with serious diseases, including amyloid-β peptide (Aβ) with Alzheimer's disease (AD), islet amyloid polypeptide (IAPP) with diabetes type 2, and prion protein (PrP) with the spongiform encephalopathies. As used herein, “amyloid,” “brain amyloid,” and “amyloid-D peptide (Aβ)” are used interchangeably.

As used herein, the term “ARIA” refers to amyloid-related imaging abnormality as evaluated using MRI. In some embodiments, ARIA includes amyloid related imaging abnormality edema/effusion (ARIA-E). In some embodiments, ARIA includes amyloid related imaging abnormality hemorrhage (ARIA-H). In some embodiments, subjects with ARIA experience headache, confusion, and/or seizure and these may be used to identify a subject with ARIA or to indicate further evaluation for ARIA. In some embodiments, ARIA is evaluated at specified intervals during treatment. In some embodiments, ARIA is evaluated when the subject experiences symptoms of ARIA.

As used herein, the term “treat” refers to obtaining one or more beneficial or desired results including, but not limited to, therapeutic benefit, by which is meant eradication or amelioration of the underlying condition being treated or of one or more of the physiological symptoms associated therewith. The term encompasses but does not require complete treatment.

As used herein, the term “prevent” refers to obtaining one or more beneficial or desired results including, but not limited to, prophylactic benefit. The benefit may include a delay or lessening of severity of symptoms of a disease, e.g., one or more symptoms of Alzheimer's disease such as progressive memory loss. For prophylactic benefit, a compound or formulation may be administered to a subject at risk of developing Alzheimer's disease, to a subject having one or more preclinical symptoms but not clinical symptoms of Alzheimer's disease, or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith.

As would be understood by one of ordinary skill in the art, digital, computerized, and/or conventional (e.g., pen and paper) cognitive tests may be used to detect early cognitive changes that may signal mild cognitive impairment and/or a risk for developing dementia, and thus may be used to identify subject in need of treatment as disclosed herein. Such tests, for example, may screen for cognitive impairment, and potentially identify subjects with MCI. Tests may use artificial intelligence to analyze cognitive test results to determine whether a case of mild cognitive impairment will escalate into Alzheimer's within a year. Diagnosing the condition early, before symptoms have begun to appear, may be used to assist physicians identify subjects in need of treatment as disclosed herein sooner, potentially delaying onset or lessening the severity of the neurodegenerative disease.

As used herein, “MMSE” refers to the Mini-Mental State Examination, a cognitive instrument commonly used for screening purposes, but also often measured longitudinally in AD clinical trials. The MMSE is a 30 point scale with higher scores indicating less impairment and lower scores indicating more impairment, ranging from 0 (most impaired) to 30 (no impairment). In some embodiments, seven items measuring orientation to time and place, registration, recall, attention, language, and drawing may be assessed as part of the MMSE score. (Folstein, M. F. et al., “Mini-mental state. A practical method for grading the cognitive state of patients for the clinician.” J. Psychiatr. Res. 1975; 12:189-98.)

As used herein, “ADAS-Cog” refers to Alzheimer's Disease Assessment Scale-Cognitive. The ADAS-Cog is a widely used cognitive scale in Alzheimer's disease trials having a structured scale that evaluates memory (word recall, delayed word recall, and word recognition), reasoning (following commands), language (naming, comprehension), orientation, ideational praxis (placing letter in envelope) and constructional praxis (copying geometric designs). (Rosen, W. G. et al., “A new rating scale for Alzheimer's disease.” Am. J. Psychiatry 1984; 141:1356-64.) Ratings of spoken language, language comprehension, word finding difficulty, ability to remember test instructions, maze, and number cancellation may also be obtained. In some embodiments, ADS-Cog refers to the use of the Alzheimer Disease Assessment Scale-Cognitive Subscale₁₁ (ADAS-Cog11). In some embodiments, ADAS-Cog₁₁ is scored from 0 to 70 points with a score of 0 indicating no impairment, and a score of 70 indicating maximum impairment. In some embodiments, ADAS-Cog refers to the use of the Alzheimer Disease Assessment Scale-Cognitive Subscale₁₄ (ADAS-Cog₁₄). ADAS-Cog 14 includes 3 additional items: maze, digit cancellation, and delayed recall tests and is scored from 0 to 90 points with a score of 0 indicating no impairment, and a score of 90 indicating maximum impairment. In some embodiments, the ADAS-Cog₁₄ tasks include memory (word recall, delayed word recall, and word recognition), reasoning (following commands), language (naming, comprehension), orientation, ideational praxis (placing letter in envelope), constructional praxis (copying geometric designs), spoken language, language comprehension, word finding difficulty, ability to remember test instructions, maze, and number cancellation (Rosen, W. G. et al., Am. J. Psychiatry 1984; 141:1356-64.).

As used herein, “CDR-SB” refers to clinical dementia rating—sum of boxes. The CDR is a clinical scale that describes 5 degrees of impairment in performance on each of 6 categories of function including memory, orientation, judgment and problem solving, community affairs, home and hobbies, and personal care. (Berg, L. et al., “Mild senile dementia of the Alzheimer type: 2. Longitudinal assessment.” Ann. Neurol. 1988; 23:477-84.) The ratings of degree of impairment obtained on each of the 6 categories of function are synthesized into 1 global rating of dementia CDR score (ranging from 0 to 3). A sum of boxes score provides an additional measure of change where each category has a maximum possible score of 3 points and the total score is a sum of the category scores giving a total possible score of 0 to 18 with higher scores indicating more impairment. The global score may be used as a clinical measure of severity of dementia.

In some embodiments, the efficacy of the treatment for Alzheimer's Disease can be measured by, for example, any one or a combination of medical observations, cognitive assessments, medical diagnostic, and medical imaging. In some embodiments, the treatment efficacy is determined by measuring tau spread via tau PET when the isolated anti-tau antibody or fragment thereof is concurrently administered with an isolated anti-Aβ protofibril antibody or fragment thereof from Week 24 to Week 104 and Week 208.

In some embodiments, treatment efficacy in symptomatic patients is assessed by measuring change from baseline after a period of treatment, e.g., a change from Week 24 to Week 208 in Clinical Dementia Rating Scale Sum of Boxes (CDR-SB) when the isolated anti-tau antibody or fragment thereof is concurrently administered with an isolated anti-Aβ protofibril antibody or fragment thereof. In some embodiments, the treatment efficacy in asymptomatic patients is assessed by measuring change in cerebrospinal fluid (CSF) phosphorylated tau (p-tau217)/total tau after a period of treatment, e.g., a change from Week 0 to Week 104 and/or Week 208 when the isolated anti-tau antibody or fragment thereof is administered alone and then concurrently administered with an isolated anti-Aβ protofibril antibody or fragment thereof.

In some embodiments, treatment efficacy is assessed in symptomatic patients by measuring at least one of the following: 1) change after a period of treatment, e.g., a change from Week 24 to Week 104 and/or Week 208 in a cognitive composite score when the isolated anti-tau antibody or isolated fragment thereof is administered in conjunction with the isolated anti-Aβ protofibril antibody or fragment thereof 2) change after a period of treatment, e.g., a change in amyloid PET from Week 0 to Week 24 to assess the effect of the isolated anti-Aβ protofibril antibody or fragment thereof when administered alone, 3) production of anti-anti-Aβ-protofibril antibodies to assess safety and tolerability when the anti-Ap-protofibril antibody or fragment thereof is administered alone after a period of treatment, e.g., a change after 24 weeks, and 4) change from Week 24 to Week 104 and/or Week 208 in CSF neurofilament light (NFL) when the anti-tau antibody or fragment thereof is administered in conjunction with the anti-Aβ protofibril antibody or fragment thereof after a period of treatment, e.g., a change from Week 24 to Week 104 and Week 208.

In some embodiments, treatment efficacy in asymptomatic patients is determined by measuring at least one of the following: 1) change after a period of treatment, e.g., a change from Week 0 to Week 52 in CSF p-tau217/total tau when the anti-tau antibody or fragment thereof is administered alone 2) change after a period of treatment, e.g., a change from Week 52 to Week 104 and/or Week 208 in CSF p-tau217)/total tau when the anti-tau antibody or fragment thereof is administered in conjunction with the anti-Aβ protofibril antibody or fragment thereof 3) the production of anti-anti-tau antibodies when the anti-tau antibody or fragment thereof is administered alone after a period of treatment, e.g., for 52 weeks, 4) change after a period of treatment, e.g., a change from Week 52 to Week 104 and/or Week 208 in CSF neurofilament light (NFL) when the anti-tau antibody or fragment thereof is administered in conjunction with the anti-Aβ protofibril antibody or fragment thereof.

### Anti-A/β Protofibril Antibody or Antigen Binding Fragment Thereof

The methods, kits, and combinations disclosed herein include an anti-Aβ protofibril antibody or antigen binding fragment thereof that is capable of binding to a human AR protofibril.

In some embodiments, the isolated anti-Aβ protofibril antibody or antigen binding fragment thereof that is capable of binding to a human Aβ protofibril comprises six CDRs (HCDR1, HCDR2, HCDR3, LCDR1, LCDR2, and LCDR3) comprising the amino acid sequences of SEQ ID NO:1 (HCDR1), SEQ ID NO:2 (HCDR2) SEQ ID NO:3 (HCDR3), SEQ ID NO: 4 (LCDR1), SEQ ID NO:5 (LCDR2), SEQ ID NO:6 (LCDR3). See, e.g., Table 11. In some embodiments, the isolated anti-Aβ protofibril antibody or antigen binding fragment thereof that is capable of binding to a human Aβ protofibril comprises six CDRs (HCDR1, HCDR2, HCDR3, LCDR1, LCDR2, and LCDR3) from a heavy chain variable domain of SEQ ID NO: 13 and a light chain variable domain of SEQ ID NO: 14 (e.g., as defined by Kabat or IMGT). In some embodiments, the anti-Aβ protofibril antibody or antigen binding fragment thereof that is capable of binding to a human Aβ protofibril comprises a heavy chain variable domain of SEQ ID NO: 13 and a light chain variable domain of SEQ ID NO:14. See, e.g., Table 12.

In some embodiments, the isolated anti-Aβ protofibril antibody or antigen binding fragment thereof that is capable of binding to a human Aβ protofibril comprises a human constant region. In some embodiments, the human constant region of the isolated anti-Aβ protofibril antibody or antigen binding fragment thereof that is capable of binding to a human Aβ protofibril comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region comprises SEQ ID NO: 17.

In some embodiments, the human constant region of the isolated anti-Aβ protofibril antibody or antigen binding fragment thereof comprises a light chain constant region chosen from κ and λ-chain constant regions and any allelic variation thereof as discussed in the Kabat report. Any one or more of such sequences may be used in the present disclosure. In some embodiments, the light chain constant region comprises SEQ ID NO: 18.

In some embodiments, the isolated anti-Aβ protofibril antibody or antigen binding fragment thereof that is capable of binding to a human Aβ protofibril is lecanemab, which is also known as BAN2401. Lecanemab is a humanized IgG1 monoclonal version of mAb158, which is a murine monoclonal antibody raised to target protofibrils and disclosed in WO 2007/108756 and Journal of Alzheimer's Disease 43: 575-588 (2015). Lecanemab is an isolated anti-Aβ protofibril antibody, demonstrating low affinity for Aβ monomer while binding with high selectivity to soluble Aβ aggregate species. For example, lecanemab has been reported demonstrates an approximately 1000-fold and 5-fold to 10-fold higher selectivity for soluble Aβ protofibrils than for Aβ monomers or Aβ-insoluble fibrils, respectively. The full length sequences of lecanemab are set forth in WO 2007/108756 and in Journal of Alzheimer's Disease 43:575-588 (2015), the disclosure of both are herein incorporated by reference.

In some embodiments, the isolated anti-Aβ protofibril antibody or antigen binding fragment thereof that is capable of binding to a human Aβ protofibril is administered twice daily. In some embodiments, the isolated anti-Aβ protofibril antibody or antigen binding fragment thereof that is capable of binding to a human Aβ protofibril is administered once daily. In some embodiments, the isolated anti-Aβ protofibril antibody or antigen binding fragment thereof that is capable of binding to a human Aβ protofibril is administered weekly. In some embodiments, the isolated anti-Aβ protofibril antibody or antigen binding fragment thereof that is capable of binding to a human Aβ protofibril is administered twice weekly. In some embodiments, the isolated anti-Aβ protofibril antibody or antigen binding fragment thereof that is capable of binding to a human Aβ protofibril is administered three times weekly. In some embodiments, the isolated anti-Aβ protofibril antibody or antigen binding fragment thereof that is capable of binding to a human Aβ protofibril is administered every 2 weeks. In some embodiments, the isolated anti-Aβ protofibril antibody or antigen binding fragment thereof that is capable of binding to a human Aβ protofibril is administered monthly.

In some embodiments, the isolated anti-Aβ protofibril antibody or antigen binding fragment thereof that is capable of binding to a human Aβ protofibril is administered at a dose ranging from 3 mg/kg to 30 mg/kg. In some embodiments, the isolated anti-Aβ protofibril antibody or antigen binding fragment thereof that is capable of binding to a human Aβ protofibril is administered at a dose of 5 mg/kg to 30 mg/kg. In some embodiments, the isolated anti-Aβ protofibril antibody or antigen binding fragment thereof that is capable of binding to a human Aβ protofibril is administered at a dose of 10 mg/kg to 30 mg/kg. In some embodiments, the isolated anti-Aβ protofibril antibody or antigen binding fragment thereof that is capable of binding to a human Aβ protofibril is administered at a dose of 15 mg/kg to 30 mg/kg. In some embodiments, the isolated anti-Aβ protofibril antibody or antigen binding fragment thereof that is capable of binding to a human Aβ protofibril is administered at a dose of 20 mg/kg to 30 mg/kg. In some embodiments, the isolated anti-Aβ protofibril antibody or antigen binding fragment thereof that is capable of binding to a human Aβ protofibril is administered at a dose of 25 mg/kg to 30 mg/kg. In some embodiments, the isolated anti-Aβ protofibril antibody or antigen binding fragment thereof that is capable of binding to a human Aβ protofibril is administered at a dose of 5 mg/kg to 25 mg/kg. In some embodiments, the isolated anti-Aβ protofibril antibody or antigen binding fragment thereof that is capable of binding to a human Aβ protofibril is administered at a dose of 5 mg/kg to 20 mg/kg. In some embodiments, the isolated anti-Aβ protofibril antibody or antigen binding fragment thereof that is capable of binding to a human Aβ protofibril is administered at a dose of 5 mg/kg to 15 mg/kg. In some embodiments, the isolated anti-Aβ protofibril antibody or antigen binding fragment thereof that is capable of binding to a human AR protofibril is administered at a dose of 5 mg/kg to 10 mg/kg. In some embodiments, the isolated anti-Aβ protofibril antibody or antigen binding fragment thereof that is capable of binding to a human Aβ protofibril is administered at a dose of 5 mg/kg, 10 mg/kg, 15 mg/kg, 20 mg/kg, 25 mg/kg, or 30 mg/kg. In some embodiments, the isolated anti-Aβ protofibril antibody or antigen binding fragment thereof that is capable of binding to a human Aβ protofibril is administered at a dose of 10 mg/kg.

In some embodiments, the isolated anti-Aβ protofibril antibody or antigen binding fragment thereof that is capable of binding to a human Aβ protofibril (e.g., lecanemab) is administered every two weeks at a dose of 10 mg/kg.

In some embodiments, the isolated anti-Aβ protofibril antibody or antigen binding fragment thereof that is capable of binding to a human Aβ protofibril is administered until a change in the level of a biomarker in a sample is observed (e.g., an increase in the Aβ42/40 ratio is observed in a plasma or CSF sample) relative to the ratio in a sample taken from the subject prior to administration. Methods for measuring the Aβ42/40 ratio are known in the art, such as assays using LC MS/MS. Methods may include the PrecivityAD™ assay (see, e.g., Kirmess et al., J. Clinica Chimica Acta 519: 267-275 (2021)) and the immunoassay using HISCL™ platform (https://www.eisai.com/news/2019/news201990.html) for measuring Aβ42 and Aβ40 in a sample to use in calculating a ratio.

In some embodiments, the biomarker is brain amyloid level, e.g., as measured by PET SUVr. Methods for calculating PET SUVr are known in the art and may include those described herein. In some embodiments, a Standard Uptake Value Ratio Quantitative analysis of amyloid levels is completed using PMOD Biomedical Image Quantification Software (PMOD Technologies, Zurich, Switzerland). In some embodiments, PET images are first assessed for subject movement in the X, Y, and Z planes and corrected for motion, if needed, before individual images (e.g., 5-minute emission frames) are averaged, e.g., using a PMOD Averaging Function (PET frames averaged to increase the signal to noise ratio). In some embodiments, corresponding MRIs from subjects are prepared (e.g., using matrix size reduction processing, cropping of the MRI to include only the brain, segmentation to separate images into binary maps of gray matter, white matter, and CSF, and stripping the image of skull leaving only brain mask). In some embodiments, the averaged PET images and prepared MRIs are matched using the PMOD Matching Function, placing the images in the same orientation. In some embodiments, a Brain Normalization function, e.g., as provided by PMOD software, is used along with Brain Norm and Rigid Matching transformation matrices, to produce an averaged PET. In some embodiments, this averaged PET which is normalized to the MNInst space (Senjem et al, 2005) that is in the same orientation as the subject's segmented MRI for quantitative analysis. In some embodiments, the PMOD Mask Function is used to mask the brain and zero the image outside of the mask to create a Normalized Gray Matter PET and a Normalized White Matter PET. Standard uptake values (SUVs) may be calculated for all gray matter mapped regions and the 3 white matter regions (pons, cerebellar white, and subcortical white) using PMOD software calculated using the normalized PET, subject weight, and injected dose of tracer to arrive at the units of SUVs. In some embodiments, the SUVr is the ratio of the global cortical average as compared to a reference region of choice. In some embodiments, a whole cerebellum mask is used as the reference region. In some embodiments, the reference region is subcortical white matter, derived whole cerebellum, whole cerebellum adjusted by subcortical white matter, cerebellar gray matter, and composite reference regions consisting of cerebellar cortex, pons subcortical white matter, and cerebella white matter.

In some embodiments, the isolated anti-Aβ protofibril antibody or antigen binding fragment thereof that is capable of binding to a human Aβ protofibril is in the form of a pharmaceutical composition. In some embodiments, the pharmaceutical composition comprising the isolated anti-Aβ protofibril antibody or antigen binding fragment thereof is administered via one or more syringes and/or autoinjectors. In some embodiments, the administration is via any suitable route, e.g., intravenous.

Lecanemab and methods comprising the use of lecanemab are disclosed in U.S. Provisional Application No. 62/749,614 and PCT International Application No. PCT/US2019/043067, both of which are incorporated herein by reference in their entireties.

Methods comprising the use of lecanemab in a subject having preclinical AD are disclosed in Clinical Trial Identifier: NCT04468659 (ClinicalTrials.gov), which are incorporated herein by reference in their entireties.

### Anti-Tau Antibody or Antigen Binding Fragment Thereof

The methods, kits, and combinations disclosed herein include an anti-tau antibody or antigen binding fragment thereof that is capable of binding to human tau.

In some embodiments, the isolated anti-tau antibody or antigen binding fragment thereof that is capable of binding to human tau comprises six CDRs (HCDR1, HCDR2, HCDR3, LCDR1, LCDR2, and LCDR3) comprising the amino acid sequences of SEQ ID NO:7 (HCDR1), SEQ ID NO:8 (HCDR2), SEQ ID NO:9 (HCDR3), SEQ ID NO:10 (LCDR1), SEQ ID NO:11 (LCDR2), and SEQ ID NO:12 (LCDR3), as defined by Kabat. In some embodiments, the isolated anti-tau antibody or antigen binding fragment thereof that is capable of binding to human tau comprises six CDRs (CDRs (HCDR1, HCDR2, HCDR3, LCDR1, LCDR2, and LCDR3) comprising the amino acid sequences of SEQ ID NO:24 (HCDR1), SEQ ID NO:25 (HCDR2), SEQ ID NO:26 (HCDR3), SEQ ID NO:27 (LCDR1), SEQ ID NO:28 (LCDR2), and SEQ ID NO:29 (LCDR3), as defined by IMGT. See, e.g., Table 11. In some embodiments, the isolated anti-tau antibody or antigen binding fragment thereof that is capable of binding to human tau comprises six CDRs (HCDR1, HCDR2, HCDR3, LCDR1, LCDR2, and LCDR3) from a heavy chain variable domain of SEQ ID NO: 15 and a light chain variable domain of SEQ ID NO: 16 (e.g., as defined by Kabat or IMGT). In some embodiments, the anti-tau antibody or antigen binding fragment thereof that is capable of binding to human tau comprises a heavy chain variable domain of SEQ ID NO: 15 and a light chain variable domain of SEQ ID NO: 16. See, e.g., Table 12.

In some embodiments, the isolated anti-tau antibody or antigen binding fragment thereof that is capable of binding to human tau comprises a human constant region. In some embodiments, the human constant region comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region comprises SEQ ID NO: 19.

In some embodiments, the human constant region of the isolated anti-tau antibody or antigen binding fragment thereof comprises a light chain constant region chosen from κ and λ-chain constant regions and any allelic variation thereof as discussed in the Kabat report. Any one or more of such sequences may be used in the present disclosure. In some embodiments, the light chain constant region comprises SEQ ID NO: 20.

In some embodiments, the anti-tau antibody or antigen binding fragment comprises E2814 or an antigen binding fragment thereof. E2814 is disclosed in US 2019/0112364 A1 as clone 7G6-HCzu25/LCzu18, the sequences of which are incorporated by reference herein.

In some embodiments, the anti-tau antibody or antigen binding fragment thereof is any of those disclosed in US 2019/0112364 A1, the disclosure of which is fully incorporated herein by reference. In some embodiments, the anti-tau antibody or antigen binding fragment thereof comprises the CDR and/or variable domain sequences from antibody clone 7G6-HCzu25/LCzu18 as disclosed in US 2019/0112364 A1, the sequences of which are incorporated by reference herein. In some embodiments, the anti-tau antibody or antigen binding fragment thereof is produced by antibody-producing cells deposited with the American Type Culture Collection (10801 University Blvd., Manassas, Va. 20110-2209) on Oct. 11, 2017 with Accession No. PTA-124524.

In some embodiments, the anti-tau antibody or antigen binding fragment thereof is administered twice daily. In some embodiments, the anti-tau antibody or antigen binding fragment thereof is administered once daily. In some embodiments, the anti-tau antibody or antigen binding fragment thereof is administered weekly. In some embodiments, the anti-tau antibody or antigen binding fragment thereof is administered twice weekly. In some embodiments, the anti-tau antibody or antigen binding fragment thereof is administered three times weekly. In some embodiments, the anti-tau antibody or antigen binding fragment thereof is administered every 2 weeks. In some embodiments, the anti-tau antibody or antigen binding fragment thereof is administered every four weeks or monthly.

In some embodiments, the anti-tau antibody or antigen binding fragment thereof is administered at a dose ranging from 200 mg to 4500 mg. In some embodiments, the anti-tau antibody or antigen binding fragment thereof is administered at a dose of 500 mg to 4500 mg. In some embodiments, the anti-tau antibody or antigen binding fragment thereof is administered at a dose of 1000 mg to 4500 mg. In some embodiments, the anti-tau antibody or antigen binding fragment thereof is administered at a dose of 1500 mg to 4500 mg. In some embodiments, the anti-tau antibody or antigen binding fragment thereof is administered at a dose of 3000 mg to 4500 mg. In some embodiments, the anti-tau antibody or antigen binding fragment thereof is administered at a dose of 200 mg to 3000 mg. In some embodiments, the anti-tau antibody or antigen binding fragment thereof is administered at a dose of 200 mg to 1500 mg. In some embodiments, the anti-tau antibody or antigen binding fragment thereof is administered at a dose of 200 mg to 1000 mg. In some embodiments, the anti-tau antibody or antigen binding fragment thereof is administered at a dose of 200 mg to 500 mg. In some embodiments, the anti-tau antibody or antigen binding fragment thereof is administered at a dose of 200 mg, 500 mg, 1000 mg, 1500 mg, 2000 mg, 2500 mg, 3000 mg, or 4500 mg. In some embodiments, the anti-tau antibody or antigen binding fragment thereof is administered at a dose of 1500 mg. In some embodiments, the anti-tau antibody or antigen binding fragment thereof is administered at a dose of 3000 mg. In some embodiments, the anti-tau antibody or antigen binding fragment thereof is administered at a dose of 4500 mg. Further information regarding the dosing and dosage forms of an anti-tau antibody or antigen binding fragment thereof that may be used in the methods disclosed herein, e.g., the anti-tau antibody E2814, can be found in International Application No. PCT/IB2022/060604, the contents of which is incorporated herein by reference in its entirety.

In some embodiments, the anti-tau antibody is E2814 or an antigen binding fragment thereof and is administered at a dose of 3000 mg, e.g. once every four weeks.

In some embodiments, the isolated anti-Aβ protofibril antibody or antigen binding fragment thereof that is capable of binding to a human Aβ protofibril is administered in conjunction with the isolated anti-tau antibody or antigen binding fragment thereof that is capable of binding to human tau, with the isolated anti-Aβ protofibril antibody or antigen binding fragment thereof that is capable of binding to a human Aβ protofibril administered every two weeks at a dose of 10 mg/kg and the isolated anti-tau antibody or antigen binding fragment thereof that is capable of binding to human tau administered every four weeks at a dose of 1500 mg or 3000 mg.

In some embodiments, the isolated anti-tau antibody or antigen binding fragment thereof that is capable of binding to human tau is administered until a change in the level of a biomarker in a sample is observed (e.g., a decrease in phosphorylated tau 217 is observed in a plasma sample) relative to the level in a sample taken from the subject prior to administration. In some embodiments, p-tau 217 can be measured in a sample taken from a subject at one point in time, and a second sample can be taken from a subject at a later point in time to measure the change in p-tau 217 in a subject. In some embodiments, the level of p-tau 217 in a sample taken from a subject after administration is reduced relative to the level of phosphorylated tau 217 in a sample taken from the subject before administration.

In some embodiments, administration of the isolated anti-tau antibody or antigen binding fragment thereof that is capable of binding to human tau is increased due to an increase in the amount of p-tau 217 in a subject. In some embodiments, administration of the isolated anti-tau antibody or antigen binding fragment thereof that is capable of binding to human tau is decreased due to a decrease in the amount of p-tau 217 in a subject. Methods for measuring phosphorylated tau 217 in plasma are known in the art, such as immunoassays. Tatebe et al., Quantification of plasma phosphorylated tau to use as a biomarker for brain Alzheimer pathology, 12 MOLECULAR NEURODEGENERATION 63 (Sep. 4, 2017).

In some embodiments, the isolated anti-tau antibody or antigen binding fragment thereof that is capable of binding to human tau is administered until a change in the spread of tau as measured by PET is detected. In some embodiments, Tau PET can be used to confirm the presence of and/or measure the amount of tau in the brain of AD subjects. In some embodiments, a tau PET scan can be taken at one point in time, and a second tau PET scan can be taken at a second point later in time to measure the spread of tau in a subject. In some embodiments, administration of the isolated anti-tau antibody or antigen binding fragment thereof that is capable of binding to human tau is increased due to tau spread as measured by PET is observed in a patient. In some embodiments, the tau PET is MK-6240 Tau PET.

### Administration

In some embodiments, the isolated anti-Aβ protofibril antibody or antigen binding fragment thereof that is capable of binding to a human Aβ protofibril is administered at the same time as the isolated anti-tau antibody or antigen binding fragment thereof that is capable of binding to human tau. In some embodiments, the isolated anti-Aβ protofibril antibody or antigen binding fragment thereof that is capable of binding to a human Aβ protofibril is administered from the same vial as the isolated anti-tau antibody or antigen binding fragment thereof that is capable of binding to human tau. In some embodiments, the isolated anti-Aβ protofibril antibody or antigen binding fragment thereof that is capable of binding to a human Aβ protofibril is administered from a different vial as the isolated anti-tau antibody or antigen binding fragment thereof that is capable of binding to human tau.

In some embodiments, the isolated anti-Aβ protofibril antibody or antigen binding fragment thereof that is capable of binding to a human Aβ protofibril is administered for a period of time before administration of the isolated anti-tau antibody or antigen binding fragment thereof that is capable of binding to human tau. In some embodiments, the isolated anti-tau antibody or antigen binding fragment thereof that is capable of binding to human tau is administered is administered for a period of time before administration of the isolated anti-Aβ protofibril antibody or antigen binding fragment thereof that is capable of binding to a human Aβ protofibril. In some embodiments, the isolated anti-tau antibody or antigen binding fragment thereof that is capable of binding to human tau is administered is administered for fifty two weeks before administration of the isolated anti-Aβ protofibril antibody or antigen binding fragment thereof that is capable of binding to a human Aβ protofibril. In some embodiments, the isolated anti-Aβ protofibril antibody or antigen binding fragment thereof that is capable of binding to a human Aβ protofibril is administered is administered for twenty four weeks before administration of the isolated anti-tau antibody or antigen binding fragment thereof that is capable of binding to human tau.

In some embodiments, the isolated anti-tau antibody or antigen binding fragment thereof that is capable of binding to human tau and the isolated anti-Aβ protofibril antibody or antigen binding fragment thereof that is capable of binding to a human Aβ protofibril are administered during the same clinical visit. In some embodiments, the isolated anti-tau antibody or antigen binding fragment thereof that is capable of binding to human tau and the isolated anti-Aβ protofibril antibody or antigen binding fragment thereof that is capable of binding to a human Aβ protofibril are administered during separate clinical visits. In some embodiments, the isolated anti-tau antibody or antigen binding fragment thereof that is capable of binding to human tau and the isolated anti-Aβ protofibril antibody or antigen binding fragment thereof that is capable of binding to a human Aβ protofibril are administered to a subject intravenously. In some embodiments, timing of administration of the isolated anti-tau antibody or antigen binding fragment thereof that is capable of binding to human tau and the isolated anti-Aβ protofibril antibody or antigen binding fragment thereof that is capable of binding to a human Aβ protofibril depends on whether a person is symptomatic for Alzheimer's disease or asymptomatic for Alzheimer's disease.

In some embodiments, a patient that is symptomatic for Alzheimer's disease is administered the isolated anti-Aβ protofibril antibody or antigen binding fragment thereof that is capable of binding to a human Aβ protofibril before being administered the isolated anti-tau antibody or antigen binding fragment thereof that is capable of binding to human tau. In some embodiments, a patient that is symptomatic for Alzheimer's disease is administered the isolated anti-Aβ protofibril antibody or antigen binding fragment thereof that is capable of binding to a human Aβ protofibril for 24 weeks until being administered the isolated anti-tau antibody or antigen binding fragment thereof that is capable of binding to human tau in conjunction with the isolated anti-Aβ protofibril antibody or antigen binding fragment thereof that is capable of binding to a human Aβ protofibril. In some embodiments, a patient that is symptomatic for Alzheimer's disease is administered the isolated anti-Aβ protofibril antibody or antigen binding fragment thereof that is capable of binding to a human Aβ protofibril for 24 weeks, then administered the isolated anti-tau antibody or antigen binding fragment thereof that is capable of binding to human tau in conjunction with the isolated anti-Aβ protofibril antibody or antigen binding fragment thereof that is capable of binding to a human Aβ protofibril from Week 24 to Week 52 and/or Week 24 to Week 208.

In some embodiments, a patient that is asymptomatic for Alzheimer's disease is administered the isolated anti-tau antibody or antigen binding fragment thereof that is capable of binding to human tau before being administered the isolated anti-Aβ protofibril antibody or antigen binding fragment thereof that is capable of binding to a human Aβ protofibril. In some embodiments, a patient that is asymptomatic for Alzheimer's disease is administered the isolated anti-tau antibody or antigen binding fragment thereof that is capable of binding to human tau for 52 weeks before being administered the isolated anti-tau antibody or antigen binding fragment thereof that is capable of binding to human tau in conjunction with the isolated anti-Aβ protofibril antibody or antigen binding fragment thereof that is capable of binding to a human Aβ protofibril. In some embodiments, a patient that is asymptomatic for Alzheimer's disease is administered the isolated anti-tau antibody or antigen binding fragment thereof that is capable of binding to human tau for 52 weeks, then administered the isolated anti-tau antibody or antigen binding fragment thereof that is capable of binding to human tau in conjunction with the isolated anti-Aβ protofibril antibody or antigen binding fragment thereof that is capable of binding to a human Aβ protofibril from Week 52 to Week 208.

### Family History and Dominantly Inherited Alzheimer's Disease

In some embodiments, the subject has a family history of Alzheimer's disease, e.g., a history of a family member being diagnosed with Alzheimer's disease before the age of 60.

In some embodiments, the subject has dominantly-inherited Alzheimer's disease (DIAD). In some embodiments, the subject has a mutation in at least one of three genes, Amyloid precursor protein (APP), Presenilin 1 (PSEN1), or Presenilin 2 (PSEN2). In some embodiments, the subject has a mutation in the APP gene. In some embodiments, the subject has a mutation in the PSEN1 gene. In some embodiments, the subject has a mutation in the PSEN2 gene. Specific mutations in the APP, PSEN1, or PSEN2 genes that contribute to DIAD are known in the art (e.g., Cruts & Van Broeckhoven, 1998; Cruts, Theuns, & Van Broeckhoven, 2012; Ryman et al., 2014; Sherva & Kowall, 2018).

### Treatment Outcomes

In some embodiments, the CDR-SB score of the subject is improved relative to the score prior to administration of the isolated anti-tau antibody or antigen binding fragment thereof that is capable of binding to human tau and/or the isolated anti-Aβ protofibril antibody or antigen binding fragment thereof that is capable of binding to a human Aβ protofibril according to the methods of treatment disclosed herein. In some embodiments, the ADAS-cog score of the subject is improved relative to the score prior to administration of the isolated anti-tau antibody or antigen binding fragment thereof that is capable of binding to human tau and/or the isolated anti-Aβ protofibril antibody or antigen binding fragment thereof that is capable of binding to a human Aβ protofibril according to the methods of treatment disclosed herein. In some embodiments, the MMSE of the subject is improved relative to the score prior to administration of the isolated anti-tau antibody or antigen binding fragment thereof that is capable of binding to human tau and/or the isolated anti-Aβ protofibril antibody or antigen binding fragment thereof that is capable of binding to a human Aβ protofibril according to the methods of treatment disclosed herein. In some embodiments, the CDR score of the subject is improved relative to the score prior to administration of the isolated anti-tau antibody or antigen binding fragment thereof that is capable of binding to human tau and/or the isolated anti-Aβ protofibril antibody or antigen binding fragment thereof that is capable of binding to a human Aβ protofibril according to the methods of treatment disclosed herein.

### Pharmaceutical Combinations and Kits

In some embodiments the isolated anti-tau antibody or antigen binding fragment thereof that is capable of binding to human tau and/or the isolated anti-Aβ protofibril antibody or antigen binding fragment thereof that is capable of binding to a human Aβ protofibril described herein are presented in the form of a pharmaceutical combination. In some embodiments the isolated anti-tau antibody or antigen binding fragment thereof that is capable of binding to human tau and/or the isolated anti-Aβ protofibril antibody or antigen binding fragment thereof that is capable of binding to a human Aβ protofibril described herein are presented in the form of a kit, e.g., comprising one or more vials comprising the antibodies or antigen binding fragments and instructions for administration, e.g., concurrent administration. In some embodiments, the antibodies or antigen binding fragments in the kit are formulated for administration, e.g., intravenous administration, e.g., formulated as disclosed above.

### Subcutaneous Administration

In some embodiments, a subject is subcutaneously administered a dose of an anti-Aβ protofibril antibody or antigen binding fragment thereof. For example, a subject may be administered from 400 mg to 800 mg or from 400 mg to 1500 mg, such as 720 mg, of the anti-Aβ protofibril antibody or antigen binding fragment thereof. The antibody may be lecanemab. The antibody may be administered at a certain frequency, e.g., twice weekly, weekly (QW), bi-weekly (every two weeks or Q2W), or monthly, for a period of time, e.g., for at least 52 weeks or 18 months, or until certain criteria is/are reached (e.g., certain behavioral and/or biomarker criteria). In some embodiments, the subject is then administered a maintenance dose of the anti-Aβ protofibril antibody or antigen binding fragment thereof that is capable of binding to a human Aβ protofibril, e.g., at a certain frequency and for a period of time or until certain criteria are reached. The dose, frequency, period of time administered, and criteria may or may not be the same as the prior treatment dose, frequency, period of time administered, and/or criteria.

In some embodiments, a treatment dose is administered weekly in two simultaneous or sequential subcutaneous injections, e.g., at a total dose of 720 mg per week. In some embodiments, treatment is for at least 52 weeks, or for at least 18 months. In some embodiments, the treatment is administered by subcutaneous injection. In some embodiments, the treatment is administered by subcutaneous autoinjector.

In some embodiments, after a period of time receiving the treatment dose (e.g., two simultaneous or sequential subcutaneous injections, e.g., at a total dose of 720 mg per week), e.g., after 18 months, a maintenance dose is administered, e.g., subcutaneously, e.g., twice weekly or weekly, e.g., at 720 mg per dose. In some embodiments, more than one treatment dose and more than one maintenance dose of the anti-Aβ protofibril antibody or antigen binding fragment thereof that is capable of binding to a human Aβ protofibril are administered, wherein the maintenance doses are administered at a lower amount and/or a reduced frequency relative to the treatment doses. In some embodiments, the criteria for switching to a maintenance dose or for selecting a lower amount or frequency of maintenance dose can include changes in biomarkers such as an increase in the Aβ42/40 ratio observed in a sample (e.g., a plasma sample) relative to the ratio in a sample taken from the subject before treatment, a decrease in a pTau217 or pTau181 level in a sample taken from the subject after treatment, and/or a reduction of amyloid PET SUVr after treatment.

In some embodiments, the anti-Aβ protofibril antibody or antigen binding fragment thereof that is capable of binding to a human Aβ protofibril is in the form of a pharmaceutical composition. In some embodiments, the pharmaceutical composition comprising the anti-Aβ protofibril antibody or antigen binding fragment thereof that is capable of binding to a human Aβ protofibril is administered via one or more syringes and/or autoinjectors. In some embodiments, the pharmaceutical composition comprising the anti-Aβ protofibril antibody or antigen binding fragment thereof that is capable of binding to a human Aβ protofibril is administered into the abdomen.

In some embodiments, the anti-Aβ protofibril antibody or antigen binding fragment thereof that is capable of binding to a human Aβ protofibril is present in a pharmaceutical composition in a concentration of at least 80 mg/mL. In some embodiments, the anti-Aβ protofibril antibody or antigen binding fragment thereof that is capable of binding to a human Aβ protofibril is present in a pharmaceutical composition in a concentration of at least 100 mg/mL. In some embodiments, the anti-Aβ protofibril antibody or antigen binding fragment thereof that is capable of binding to a human Aβ protofibril is present in a pharmaceutical composition in a concentration of at least 200 mg/mL. In some embodiments, the anti-Aβ protofibril antibody or antigen binding fragment thereof that is capable of binding to a human Aβ protofibril is present in a pharmaceutical composition in a concentration of at least 250 mg/mL. In some embodiments, the antibody is present in a pharmaceutical composition in a concentration of 80 mg/mL to 300 mg/mL. In some embodiments, the anti-Aβ protofibril antibody or antigen binding fragment thereof that is capable of binding to a human Aβ protofibril is present in a pharmaceutical composition in a concentration of 85 mg/mL to 275 mg/mL. In some embodiments, the anti-Aβ protofibril antibody or antigen binding fragment thereof that is capable of binding to a human Aβ protofibril is present in a pharmaceutical composition in a concentration of 90 mg/mL to 250 mg/mL. In some embodiments, the anti-Aβ protofibril antibody or antigen binding fragment thereof that is capable of binding to a human Aβ protofibril is present in a pharmaceutical composition in a concentration of 95 mg/mL to 225 mg/mL. In some embodiments, the anti-Aβ protofibril antibody or antigen binding fragment thereof that is capable of binding to a human Aβ protofibril is present in a pharmaceutical composition in a concentration of 100 mg/mL to 200 mg/mL. In some embodiments, the anti-Aβ protofibril antibody or antigen binding fragment thereof that is capable of binding to a human Aβ protofibril is present in a pharmaceutical composition in a concentration of 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, or 300 mg/mL. In some embodiments, the anti-Aβ protofibril antibody or antigen binding fragment thereof that is capable of binding to a human Aβ protofibril is present in a pharmaceutical composition in a concentration of 100 mg/mL. In some embodiments, the anti-Aβ protofibril antibody or antigen binding fragment thereof that is capable of binding to a human Aβ protofibril is present in a pharmaceutical composition in a concentration of 200 mg/mL. In some embodiments, the anti-Aβ protofibril antibody or antigen binding fragment thereof that is capable of binding to a human Aβ protofibril is present in a pharmaceutical composition in a concentration of 250 mg/mL. In some embodiments, the anti-Aβ protofibril antibody or antigen binding fragment thereof that is capable of binding to a human Aβ protofibril is present in a pharmaceutical composition in a concentration of 300 mg/mL. In some embodiments, the anti-Aβ protofibril antibody or antigen binding fragment thereof that is capable of binding to a human Aβ protofibril is lecanemab.

In some embodiments, the pharmaceutical composition comprising an anti-Aβ protofibril antibody or antigen binding fragment thereof further comprises at least one additional component. In some embodiments, the at least one additional component in the pharmaceutical composition is chosen from pharmaceutically acceptable buffers. In some embodiments, the pharmaceutically acceptable buffer is a citrate buffer. In some embodiments, the pharmaceutically acceptable buffer is a histidine buffer. In some embodiments, the at least one additional component in the pharmaceutical composition is chosen from emulsifiers. In some embodiments, the at least one additional component in the pharmaceutical composition is chosen from citric acid (or citric acid monohydrate), sodium chloride, histidine (and/or histidine hydrochloride), arginine (and/or arginine hydrochloride), and polysorbate 80. In some embodiments, the at least one additional component in the pharmaceutical composition is chosen from citric acid (and/or citric acid monohydrate), arginine (and/or arginine hydrochloride), and polysorbate 80. In some embodiments, the at least one additional component in the pharmaceutical composition is chosen from histidine (and/or histidine hydrochloride), arginine (and/or arginine hydrochloride), and polysorbate 80.

In some embodiments, the pharmaceutical composition comprising the anti-Aβ protofibril antibody or antigen binding fragment thereof comprises arginine (and/or arginine hydrochloride). In some embodiments, the concentration of arginine (and/or arginine hydrochloride) in the pharmaceutical composition ranges from 100 mM to 400 mM. In some embodiments, the concentration of arginine (and/or arginine hydrochloride) in the pharmaceutical composition ranges from 110 mM to 380 mM, 120 mM to 360 nM, 125 nM to 350 mM, 140 mM to 340 mM, 160 mM to 325 mM, 175 mM to 300 mM, or 200 mM to 250 mM. In some embodiments, the concentration of arginine (and/or arginine hydrochloride) in the pharmaceutical composition ranges from 110 mM to 150 mM, 150 mM to 200 mM, 200 mM to 250 mM, 250 mM to 300 mM, 300 mM to 350 mM, or 350 mM to 380 mM. In some embodiments, the concentration of arginine (and/or arginine hydrochloride) is 125 mM. In some embodiments, the concentration of arginine (and/or arginine hydrochloride) is 200 mM. In some embodiments, the concentration of arginine (and/or arginine hydrochloride) is 350 mM.

In some embodiments, the pharmaceutical composition comprising the anti-Aβ protofibril antibody or antigen binding fragment thereof comprises histidine. In some embodiments, the concentration of histidine in the pharmaceutical composition ranges from 10 mM to 100 mM. In some embodiments, the concentration of histidine in the pharmaceutical composition ranges from 10 mM to 100 mM, 12 mM to 80 mM, 14 mM to 60 mM, 15 mM to 55 mM, 15 mM to 35 mM, or 15 mM to 25 mM. In some embodiments, the concentration of histidine is 25 mM. In some embodiments, the concentration of histidine is 50 mM.

In some embodiments, the pharmaceutical composition comprising the anti-Aβ protofibril antibody or antigen binding fragment thereof comprises polysorbate 80. In some embodiments, the concentration of polysorbate 80 in the pharmaceutical composition ranges from 0.01 to 0.1% w/v, 0.01 to 0.08% w/v, 0.02 to 0.08% w/v, 0.03 to 0.07% w/v, or 0.04 to 0.06% w/v. In some embodiments, the polysorbate 80 is present in the pharmaceutical composition in a concentration of 0.01% w/v, 0.02% w/v, 0.03% w/v, 0.04% w/v, 0.05% w/v, 0.06% w/v, 0.07% w/v, or 0.08% w/v. In some embodiments, the polysorbate 80 is present in the pharmaceutical composition in a concentration of 0.02% w/v. In some embodiments, the polysorbate 80 is present in the pharmaceutical composition in a concentration of 0.05% w/v.

In some embodiments, the pharmaceutical composition comprising the anti-Aβ protofibril antibody or antigen binding fragment thereof comprises citric acid monohydrate. In some embodiments, the concentration of citric acid monohydrate in the pharmaceutical composition ranges from 10 mM to 100 mM. In some embodiments, the concentration of citric acid monohydrate in the pharmaceutical composition ranges from 10 mM to 100 mM, 10 mM to 90 mM, 15 mM to 85 mM, 20 mM to 80 mM, 25 mM to 75 mM, 30 mM to 70 mM, 30 mM to 60 mM, or 30 mM to 50 mM. In some embodiments, the concentration of citric acid monohydrate in the pharmaceutical composition is 50 mM.

In some embodiments, the pharmaceutical composition comprising the anti-Aβ protofibril antibody or antigen binding fragment thereof has a pH in the range of 4.5 to 5.5. In some embodiments, the pH in the pharmaceutical composition is in the range of 4.0 to 6.0, 4.2 to 5.8, 4.3 to 5.7, 4.4 to 5.6, or 4.5 to 5.5. In some embodiments, the pH is 4.5, 4.6, 4.7, 4.8, 4.9, 5.0, 5.1, 5.2, 5.3, 5.4 or 5.5. In some embodiments, the pH is 5.0.

In some embodiments, the pharmaceutical compositions comprising the anti-Aβ protofibril antibody or antigen binding fragment thereof may be in the form of a solution and/or any other suitable liquid formulation deemed appropriate by one of ordinary skill in the art. In some embodiments, the pharmaceutical composition is formulated as a sterile, non-pyrogenic liquid for subcutaneous administration. In some embodiments, the pharmaceutical composition is a saline solution.

In some embodiments, the pharmaceutical composition comprising the anti-Aβ protofibril antibody or antigen binding fragment thereof is a liquid dosage form comprising an anti-Aβ protofibril antibody or antigen binding fragment thereof that is capable of binding to a human Aβ protofibril that binds to Aβ protofibril, such as lecanemab, and further comprising, for instance, citric acid monohydrate, arginine, arginine hydrochloride, and polysorbate 80.

In some embodiments, the pharmaceutical composition comprises 100 mg/mL of the anti-Aβ protofibril antibody or antigen binding fragment thereof, such as lecanemab, 50 mM citric acid monohydrate, 110 mM arginine, 240 mM arginine hydrochloride, and 0.05% (w/v) polysorbate 80, and has a pH of 5.0±0.4.

In some embodiments, the pharmaceutical composition comprising the anti-Aβ protofibril antibody or antigen binding fragment thereof is a liquid dosage form comprising an anti-Aβ protofibril antibody that binds to Aβ protofibril, such as lecanemab, and further comprising, for instance, histidine, histidine hydrochloride, arginine hydrochloride, and polysorbate 80. In some embodiments, the pharmaceutical composition comprises 100 mg/mL or 200 mg/mL of an anti-Aβ protofibril antibody that binds to Aβ protofibril, such as lecanemab, 25 mM of histidine and histidine hydrochloride, 200 mM arginine hydrochloride, and 0.05% (w/v) polysorbate 80, and has a pH of 5.0±0.4. In some embodiments, the pharmaceutical composition comprises as a sterile aqueous solution 200 mg/mL lecanemab, 200 mM arginine, 25 mM histidine and histidine hydrochloride, 0.05% (w/v) Polysorbate 80.

In some embodiments, the pharmaceutical composition is a liquid dosage form comprising an anti-Aβ protofibril antibody that binds to Aβ protofibril, such as lecanemab, and further comprising, for instance, histidine, histidine hydrochloride, arginine hydrochloride, and polysorbate 80. In some embodiments, the pharmaceutical composition comprises 200 mg/mL of an anti-Aβ protofibril antibody that binds to Aβ protofibril, such as lecanemab, 50 mM histidine and histidine hydrochloride, 125 mM arginine hydrochloride, and 0.02% (w/v) polysorbate 80, and has a pH of 5.0±0.4.

In some embodiments, the pharmaceutical composition is a liquid dosage form comprising an anti-Aβ protofibril antibody that binds to Aβ protofibril, such as lecanemab, and further comprising, for instance, histidine, histidine hydrochloride, arginine hydrochloride, and polysorbate 80. In some embodiments, the pharmaceutical composition comprises 200 mg/mL of an anti-Aβ protofibril antibody that binds to Aβ protofibril, such as lecanemab, 50 mM citric acid (and/or citric acid monohydrate), 125 mM arginine (and/or arginine hydrochloride), and 0.02% (w/v) polysorbate 80, and has a pH of 5.0±0.4.

Exemplary formulations for subcutaneous administration are also disclosed in PCT/IB2021/000155, the contents of which are incorporated by reference herein in their entirety.

As used herein, the term “maintenance dose” refers to a dosage administered to a subject to maintain the desired therapeutic effect. In some embodiments, a subject's maintenance dose is the same as the dose during the treatment period. In some embodiments, the maintenance dose is administered subcutaneously. In some embodiments, the maintenance dose is administered once or multiple times. In some embodiments, the maintenance dose is administered weekly, every two weeks, every 4 weeks, every 6 weeks, every 8 weeks, every 10 weeks, every 12 weeks (every three months or quarterly), every 16 weeks, every 24 weeks (every six months or semi-annually), every 48 weeks, monthly, every 2 months, every 3 months, every 4 months, every 6 months, or every 12 months. In some embodiments, the maintenance dose comprises an anti-Aβ protofibril antibody or antigen binding fragment thereof that is capable of binding to a human Aβ protofibril. In some embodiments, the maintenance dose is 300 mg to 800 mg, 300 mg to 400 mg, 400 mg to 500 mg, 400 mg to 450 mg, 450 mg to 500 mg, 500 mg to 600 mg, 500 mg to 550 mg, 550 mg to 600 mg, 600 mg to 700 mg, 600 mg to 650 mg, 650 mg to 700 mg, 700 mg to 800 mg, 700 mg to 750 mg, or 750 mg to 800 mg. In some embodiments, the maintenance dose is 300 mg, 310 mg, 320 mg, 330 mg, 340 mg, 350 mg, 360 mg, 370 mg, 380 mg, or 390 mg. In some embodiments, the maintenance dose is 400 mg, 410 mg, 420 mg, 430 mg, 440 mg, 450 mg, 460 mg, 470 mg, 480 mg, or 490 mg. In some embodiments, the maintenance dose is 500 mg, 510 mg, 520 mg, 530 mg, 540 mg, 550 mg, 560 mg, 570 mg, 580 mg, or 590 mg. In some embodiments, the maintenance dose is 600 mg, 610 mg, 620 mg, 630 mg, 640 mg, 650 mg, 660 mg, 670 mg, 680 mg, or 690 mg. In some embodiments, the maintenance dose is 700 mg, 710 mg, 720 mg, 730 mg, 740 mg, 750 mg, 760 mg, 770 mg, 780 mg, or 790 mg. In some embodiments, the maintenance dose is 800 mg to 1600 mg, 800 mg to 1000 mg, 800 mg to 900 mg, 900 mg to 1000 mg, 1000 mg to 1200 mg, 1000 mg tov 1100 mg, 1100 mg to 1200 mg, 1200 mg to 1400 mg, 1200 mg to 1300 mg, 1300 mg to 1400 mg, 1400 mg to 1600 mg, 1400 mg to 1500 mg, or 1500 mg to 16000 mg. In some embodiments, the maintenance dose is 800 mg, 820 mg, 840 mg, 860 mg, 880 mg, 900 mg, 920 mg, 940 mg, 960 mg, or 980 mg. In some embodiments, the maintenance dose is 1000 mg, 1020 mg, 1040 mg, 1060 mg, 1080 mg, 1100 mg, 1120 mg, 1140 mg, 1160 mg, or 1180 mg. In some embodiments, the maintenance dose is 1200 mg, 1220 mg, 1240 mg, 1260 mg, 1280 mg, 1300 mg, 1320 mg, 1340 mg, 1360 mg, or 1380 mg. In some embodiments, the maintenance dose is 1400 mg, 1420 mg, 1440 mg, 1460 mg, 1480 mg, 1500 mg, 1520 mg, 1540 mg, 1560 mg, or 1580 mg. In some embodiments, the maintenance dose is provided in a single administration, e.g., administered as a single subcutaneous injection of 1440 mg, or in two or more administrations, two administrations of 720 mg for a total of 1440 mg, four administrations of 360 mg for a total of 1440 mg. In some embodiments, the maintenance dose is 3600 mg. In some embodiments, the maintenance dose is 440 mg. In some embodiments, the maintenance dose is 580 mg. In some embodiments, the maintenance dose is 720 mg. In some embodiments, the maintenance dose of 720 mg is provided in a single administration or in two administrations of 360 mg. In some embodiments, the maintenance dose is 1440 mg. In some embodiments, the maintenance dose is provided in a single administration, e.g., administered as a single subcutaneous injection of 720 or 1440 mg, or in two or more administrations, e.g., two concurrent administrations of 360 mg for a total of 720 mg or two administrations of 720 mg for a total of 1440 mg. or four administrations of 360 mg for a total of 1440 mg. In some embodiments, the maintenance dose is 120 mg. In some embodiments, the maintenance dose is 180 mg. In some embodiments, the maintenance dose is 240 mg. In some embodiments, the maintenance dose is 360 mg. In some embodiments, the maintenance dose is 440 mg. In some embodiments, the maintenance dose is 480 mg. In some embodiments, the maintenance dose is 540 mg. In some embodiments, the maintenance dose is 440 mg. In some embodiments, the maintenance dose is 580 mg. In some embodiments, the maintenance dose is 600 mg. In some embodiments, the maintenance dose is 720 mg. In some embodiments, the maintenance dose is 840 mg. In some embodiments, the maintenance dose is 900 mg. In some embodiments, the maintenance dose is 960 mg. In some embodiments, the maintenance dose is 1080 mg. In some embodiments, the maintenance dose is 1200 mg. In some embodiments, the maintenance dose is 1260 mg. In some embodiments, the maintenance dose is 1320 mg. In some embodiments, the maintenance dose is 1440 mg. In some embodiments, the maintenance dose is administered as a weekly, subcutaneous injection of 720 mg. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 1440 mg. In some embodiments, the maintenance dose is provided in a single, biweekly administration of 1440 mg comprising two concurrent, e.g., two sequential administrations of 720 mg of the subcutaneous formulation for a total of 1440 mg or four sequential administrations of 360 mg for a total of 1440 mg.

In some embodiments, the maintenance dose is administered once or multiple times. In some embodiments, the maintenance dose is administered at a lower dose than during an earlier course of treatment and/or is administered less frequently than during the earlier course of treatment.

In some embodiments, the treatment dose is administered as a subcutaneous injection. In some embodiments, the treatment dose is administered as a weekly, subcutaneous injection. In some embodiments, the treatment dose is administered as a biweekly, subcutaneous injection. In some embodiments, the treatment dose is administered as a monthly, subcutaneous injection. In some embodiments, the treatment dose is administered as a quarterly, subcutaneous injection.

In some embodiments, the maintenance dose is administered as a subcutaneous injection. In some embodiments, the maintenance dose is administered as a weekly, subcutaneous injection. In some embodiments, the maintenance dose is administered as a biweekly, subcutaneous injection. In some embodiments, the maintenance dose is administered as a monthly, subcutaneous injection. In some embodiments, the maintenance dose is administered as a quarterly, subcutaneous injection.

In some embodiments, the frequency of the maintenance dose is every week. In some embodiments, the maintenance dose is every two weeks (bi-weekly). In some embodiments, the maintenance dose is every four weeks (monthly). In some embodiments, the subcutaneous maintenance dose is administered every six weeks. In some embodiments, the subcutaneous maintenance dose is administered every eight weeks (2 months). In some embodiments, the maintenance dose is every three months (every twelve weeks or quarterly). In some embodiments, the maintenance dose is every six months (every 24 weeks or semi-annually). In some embodiments, a subject's maintenance dose is the same as the dose during the treatment period. In some embodiments, the maintenance dose is same dose amount as the dose prior to administering the maintenance dose. In some embodiments, the maintenance dose amount is lower dose than the dose prior to administering the maintenance dose. In some embodiments, the maintenance dose is same dose frequency as the dose prior to administering the maintenance dose. In some embodiments, the maintenance dose is lower dose frequency than the dose prior to administering the maintenance dose.

In some embodiments, the maintenance dose is administered as a subcutaneous injection of the anti-Aβ protofibril antibody or antigen binding fragment thereof that is capable of binding to a human Aβ protofibril. In some embodiments, the maintenance dose is administered as a weekly subcutaneous injection of the subcutaneous formulation of the anti-Aβ protofibril antibody or antigen binding fragment thereof that is capable of binding to a human Aβ protofibril. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the subcutaneous maintenance dose is administered weekly. In some embodiments, the subcutaneous maintenance dose is administered every two weeks. In some embodiments, the subcutaneous maintenance dose is administered every four weeks (monthly). In some embodiments, the subcutaneous maintenance dose is administered every six weeks. In some embodiments, the subcutaneous maintenance dose is administered every eight weeks (2 months). In some embodiments, the subcutaneous maintenance dose is administered every three months (twelve weeks or quarterly). In some subcutaneous embodiments, the maintenance dose is administered weekly, every two weeks, every 4 weeks, every 6 weeks, every 8 weeks, every 10 weeks, every 12 weeks, every 16 weeks, every 24 weeks, every 48 weeks, monthly, every 2 months, every 3 months, every 4 months, every 6 months, or every 12 months. In some embodiments, the subcutaneous maintenance dose comprises an anti-Aβ protofibril antibody or antigen binding fragment thereof that is capable of binding to a human Aβ protofibril at a dose of 300 mg to 800 mg, 300 mg to 400 mg, 400 mg to 500 mg, 400 mg to 450 mg, 450 mg to 500 mg, 500 mg to 600 mg, 500 mg to 550 mg, 550 mg to 600 mg, 600 mg to 700 mg, 600 mg to 650 mg, 650 mg to 700 mg, 700 mg to 800 mg, 700 mg to 750 mg, or 750 mg to 800 mg. In some embodiments, the maintenance dose is 300 mg, 310 mg, 320 mg, 330 mg, 340 mg, 350 mg, 360 mg, 370 mg, 380 mg, or 390 mg. In some embodiments, the maintenance dose is 400 mg, 410 mg, 420 mg, 430 mg, 440 mg, 450 mg, 460 mg, 470 mg, 480 mg, or 490 mg. In some embodiments, the maintenance dose is 500 mg, 510 mg, 520 mg, 530 mg, 540 mg, 550 mg, 560 mg, 570 mg, 580 mg, or 590 mg. In some embodiments, the maintenance dose is 600 mg, 610 mg, 620 mg, 630 mg, 640 mg, 650 mg, 660 mg, 670 mg, 680 mg, or 690 mg. In some embodiments, the maintenance dose is 700 mg, 710 mg, 720 mg, 730 mg, 740 mg, 750 mg, 760 mg, 770 mg, 780 mg, or 790 mg. In some embodiments, the maintenance dose is 800 mg to 1600 mg, 800 mg to 1000 mg, 800 mg to 900 mg, 900 mg to 1000 mg, 1000 mg to 1200 mg, 1000 mg to 1100 mg, 1100 mg to 1200 mg, 1200 mg to 1400 mg, 1200 mg to 1300 mg, 1300 mg to 1400 mg, 1400 mg to 1600 mg, 1400 mg to 1500 mg, or 1500 mg to 16000 mg. In some embodiments, the maintenance dose is 800 mg, 820 mg, 840 mg, 860 mg, 880 mg, 900 mg, 920 mg, 940 mg, 960 mg, or 980 mg. In some embodiments, the maintenance dose is 1000 mg, 1020 mg, 1040 mg, 1060 mg, 1080 mg, 1100 mg, 1120 mg, 1140 mg, 1160 mg, or 1180 mg. In some embodiments, the maintenance dose is 1200 mg, 1220 mg, 1240 mg, 1260 mg, 1280 mg, 1300 mg, 1320 mg, 1340 mg, 1360 mg, or 1380 mg. In some embodiments, the maintenance dose is 1400 mg, 1420 mg, 1440 mg, 1460 mg, 1480 mg, 1500 mg, 1520 mg, 1540 mg, 1560 mg, or 1580 mg. In some embodiments, the maintenance dose is provided in a single administration, e.g., administered as a single subcutaneous injection of 720 or 1440 mg, or in two or more administrations, e.g., two concurrent administrations of 360 mg for a total of 720 mg or two administrations of 720 mg for a total of 1440 mg, or four administrations of 360 mg for a total of 1440 mg. In some embodiments, the maintenance dose is 440 mg. In some embodiments, the maintenance dose is 580 mg. In some embodiments, the maintenance dose is administered as a single administration of 720 mg or two administrations of 360 mg. In some embodiments, the maintenance dose is 1440 mg. In some embodiments, the maintenance dose is administered as a weekly, subcutaneous injection of 720 mg. In some embodiments, the maintenance dose is administered as a weekly, subcutaneous injection of 360 mg. In some embodiments, the maintenance dose is administered as a biweekly, subcutaneous injection of 720 mg. In some embodiments, the maintenance dose is administered as a biweekly, subcutaneous injection of 1440 mg. In some embodiments, the maintenance dose is provided in a single, biweekly administration of 1440 mg comprising two concurrent, e.g., sequential administrations of 720 mg of the subcutaneous formulation for a total of 1440 mg.

In some embodiments, a treatment comprises subcutaneously administering an anti-Aβ protofibril antibody or antigen binding fragment thereof that is capable of binding to a human Aβ protofibril before switching to an intravenous maintenance dose. In some embodiments, a treatment comprises subcutaneously administering lecanemab weekly, e.g., a subcutaneous injection of 720 mg comprising two concurrent, e.g., sequential, injections of 360 mg (2×1.8 mL of 400 mg/2 mL), e.g., until a patient is amyloid-negative or e.g., for a period of time, e.g., at least 18 months. In some embodiments, a treatment comprises subcutaneously administering lecanemab weekly, e.g., at a dose of 720 mg, e.g., for at least 18 months or e.g., until a patient is amyloid-negative, and then switching to a maintenance dose. In some embodiments, a treatment comprises subcutaneously administering lecanemab weekly, e.g., at a dose of 720 mg, e.g., for at least 18 months or e.g., until a patient is amyloid-negative, before switching to an intravenous maintenance dose of 10 mg/kg weekly. In some embodiments, a treatment comprises subcutaneously administering lecanemab weekly, e.g., at a dose of 720 mg, e.g., for at least 18 months or e.g., until a patient is amyloid-negative, before switching to an intravenous maintenance dose of 10 mg/kg biweekly. In some embodiments, a treatment comprises subcutaneously administering lecanemab weekly, e.g., at a dose of 720 mg, e.g., for at least 18 months or e.g., until a patient is amyloid-negative, before switching to an intravenous maintenance dose of 10 mg/kg monthly. In some embodiments, a treatment comprises subcutaneously administering lecanemab weekly, e.g., at a dose of 720 mg, e.g., for at least 18 months or e.g., until a patient is amyloid-negative, before switching to an intravenous maintenance dose of 10 mg/kg every six weeks. In some embodiments, a treatment comprises subcutaneously administering lecanemab weekly, e.g., at a dose of 720 mg, e.g., for at least 18 months or e.g., until a patient is amyloid-negative, before switching to an intravenous maintenance dose of 10 mg/kg every eight weeks. In some embodiments, a treatment comprises subcutaneously administering lecanemab weekly, e.g., at a dose of 720 mg, e.g., for at least 18 months or e.g., until a patient is amyloid-negative, before switching to an intravenous maintenance dose of 10 mg/kg quarterly. In some embodiments, a subject's maintenance dose is administered at the same amount and/or frequency as the dose during the treatment period. In some embodiments, a subject's maintenance dose is 50% of the dose during the treatment period.

In some embodiments, the maintenance dose is administered intravenously, e.g., after an intravenous treatment period as disclosed above. In some embodiments, an intravenous maintenance dose, e.g., a dosing of 10 mg/kg lecanemab, is administered every week, two weeks, every month, every two months, or every three months (quarterly). In some embodiments, the intravenous maintenance dose is administered every two weeks. In some embodiments, the intravenous maintenance dose is administered every four weeks. In some embodiments, the intravenous maintenance dose is administered every six weeks. In some embodiments, the intravenous maintenance dose is administered every eight weeks (2 months). In some embodiments, the intravenous maintenance dose is administered every three months (quarterly). In some embodiments, the intravenous maintenance dose is administered every 24 weeks (every six months or semi-annually). In some embodiments, the intravenous maintenance dose is 2.5 mg/kg-10 mg/kg. In some embodiments, the maintenance dose is administered as a biweekly, intravenous dose of 10 mg/kg lecanemab. In some embodiments, the maintenance dose is administered as an intravenous dose of 10 mg/kg every four weeks (monthly). In some embodiments, the maintenance dose is administered as an intravenous dose of 10 mg/kg every six weeks. In some embodiments, the maintenance dose is administered as an intravenous dose of 10 mg/kg every eight weeks (2 months). In some embodiments, the maintenance dose is administered as an intravenous dose of 10 mg/kg every twelve weeks (every three months or quarterly). In some embodiments, the maintenance dose is administered as an intravenous dose of 10 mg/kg every 24 weeks (every six months or semi-annually). In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a weekly intravenous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a biweekly intravenous maintenance dose. In some embodiments, a treatment comprises administering intravenously an anti-Aβ protofibril antibody or antigen binding fragment thereof that is capable of binding to a human AR protofibril at 10 mg/kg, biweekly, e.g., for at least 18 months or e.g., until a patient is amyloid-negative, before switching to a monthly intravenous maintenance dose. In some embodiments, a treatment comprises administering intravenously an anti-Aβ protofibril antibody or antigen binding fragment thereof that is capable of binding to a human AR protofibril at 10 mg/kg, biweekly, e.g., for at least 18 months or e.g., until a patient is amyloid-negative, before switching to an intravenous maintenance dose every six weeks. In some embodiments, a treatment comprises administering intravenously an anti-Aβ protofibril antibody or antigen binding fragment thereof that is capable of binding to a human AR protofibril at 10 mg/kg, biweekly, e.g., for at least 18 months or e.g., until a patient is amyloid-negative, before switching to an intravenous maintenance dose every eight weeks. In some embodiments, a treatment comprises administering intravenously an anti-Aβ protofibril antibody or antigen binding fragment thereof that is capable of binding to a human AR protofibril at 10 mg/kg, biweekly, e.g., for at least 18 months or e.g., until a patient is amyloid-negative, before switching to a quarterly intravenous maintenance dose.

In some embodiments, a patient starts on an intravenous maintenance dose, e.g., a dosing of 10 mg/kg lecanemab as disclosed above before switching to a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation. In some embodiments, a patient starts on a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation before switching to an intravenous maintenance dose, e.g., a dosing of 10 mg/kg lecanemab as disclosed above.

In some embodiments, the anti-Aβ protofibril antibody or antigen binding fragment thereof that is capable of binding to a human Aβ protofibril is administered subcutaneously (SC). In some embodiments, the anti-Aβ protofibril antibody or antigen binding fragment thereof that is capable of binding to a human Aβ protofibril is administered in an injection having a volume of 1.1 mL. In some embodiments, the anti-Aβ protofibril antibody or antigen binding fragment thereof that is capable of binding to a human AR protofibril is administered in an injection having a volume of 1.4 mL. In some embodiments, the anti-Aβ protofibril antibody or antigen binding fragment thereof that is capable of binding to a human Aβ protofibril is administered in an injection having a volume of 1.45 mL. In some embodiments, the anti-Aβ protofibril antibody or antigen binding fragment thereof that is capable of binding to a human Aβ protofibril is administered in an injection having a volume of 1.8 mL.

In some embodiments, the anti-Aβ protofibril antibody or antigen binding fragment thereof that is capable of binding to a human Aβ protofibril is administered once daily. In some embodiments, the anti-Aβ protofibril antibody or antigen binding fragment thereof that is capable of binding to a human Aβ protofibril is administered twice daily. In some embodiments, the anti-Aβ protofibril antibody or antigen binding fragment thereof that is capable of binding to a human Aβ protofibril is administered once or multiple times; for example, the anti-Aβ protofibril antibody or antigen binding fragment thereof that is capable of binding to a human Aβ protofibril is administered as a single an administration of 720 mg or as two administrations of 720 mg for a total of 1440 mg. In some embodiments, the anti-Aβ protofibril antibody or antigen binding fragment thereof that is capable of binding to a human Aβ protofibril is administered once or multiple times; for example, the anti-Aβ protofibril antibody or antigen binding fragment thereof that is capable of binding to a human Aβ protofibril is administered as a single administration of 720 mg or as two administrations of 360 mg for a total of 720 mg. In some embodiments, the anti-Aβ protofibril antibody or antigen binding fragment thereof that is capable of binding to a human Aβ protofibril is administered weekly. In some embodiments, the anti-Aβ protofibril antibody or antigen binding fragment thereof that is capable of binding to a human Aβ protofibril is administered twice weekly. In some embodiments, the anti-Aβ protofibril antibody or antigen binding fragment thereof that is capable of binding to a human Aβ protofibril is administered three times weekly. In some embodiments, the anti-Aβ protofibril antibody or antigen binding fragment thereof that is capable of binding to a human Aβ protofibril is administered every 2 weeks. In some embodiments, the anti-Aβ protofibril antibody or antigen binding fragment thereof that is capable of binding to a human Aβ protofibril is administered monthly. In some embodiments, the dose amount and/or the dose frequency may be reduced after the desired therapeutic effect is achieved. The reduced frequency may be every two weeks, or every 4 weeks, every 6 weeks, every 8 weeks, every 10 weeks, every 12 weeks, every 16 weeks, monthly, every 2 months, every 3 months, every 4 months, every 6 months, or every 12 months. In some embodiments, the desired therapeutic effect that related to the reduction of the dose amount or the dose frequency may be one or more selected from reduction of brain amyloid, reduction of amyloid PET SUVr, increase of plasma Aβ42/40 ratio, reduction of plasma p-tauT81, and changes in other biomarkers correlating with brain amyloid reduction, that achieve sufficient or predetermined levels. In some embodiments, the administration of the anti-Aβ protofibril antibody or antigen binding fragment thereof that is capable of binding to a human Aβ protofibril is discontinued when the desired therapeutic effect is maintained after the reduction of the dose amount or the dose frequency. In some embodiments, the administration of the anti-Aβ protofibril antibody or antigen binding fragment thereof that is capable of binding to a human Aβ protofibril is discontinued if the desired therapeutic effect, which may be evaluated by one or more of selected from reduction of brain amyloid, reduction of amyloid PET SUVr, increase of plasma Aβ42/40 ratio, reduction of plasma p-tau181, and changes in other biomarkers correlating with brain amyloid reduction, is not achieved or expected sufficient or predetermined levels in a subject.

In some embodiments, a treatment comprises subcutaneously administering lecanemab weekly, e.g., at a dose of 720 mg, e.g., for at least 18 months. In some embodiments, a treatment comprises administering subcutaneously lecanemab twice weekly, e.g., at 720 mg per dose, e.g., for at least 18 months. In some embodiments, treatment is continued until a desired improvement in one or more biomarker or other treatment outcome measure is achieved, e.g., when an increase in the Aβ42/40 ratio is observed in a sample (e.g., a plasma sample) relative to the ratio in a sample taken from the subject before treatment, e.g., before 18 months of treatment. In some embodiments, the subject has been diagnosed with early AD. In some embodiments, the subject has been diagnosed as having mild cognitive impairment due to Alzheimer's disease—intermediate likelihood and/or has been diagnosed as having mild Alzheimer's disease dementia.

In some embodiments, the method of treatment comprises measuring the concentration of amyloid β 1-42 (Aβ42) and a concentration of amyloid β 1-40 (Aβ40) in a first blood sample obtained from the subject to determine a first ratio of Aβ42 to Aβ40 (Aβ42/40 ratio). In some embodiments, the subject is then administered a therapeutically effective dose of an anti-amyloid 3 (AD) protofibril antibody. In some embodiments, a second blood sample is obtained after the first sample to determine a second Aβ42/40 ratio. In some embodiments, a second blood sample is obtained from a subject after treatment has stopped or been reduced. In some embodiments, a change in the Aβ42/40 ratio is used to determine a second therapeutically effective dose. In some embodiments, a subject having an elevated second ratio relative to the first ratio is administered a second therapeutically effective dose comprising the same or a lower amount of the anti-Aβ protofibril antibody or antigen binding fragment thereof that is capable of binding to a human Aβ protofibril than in the first dose to the subject. In some embodiments, a subject having a lower second ratio relative to the first ratio is administered a second therapeutically effective dose comprising a higher amount of the anti-Aβ protofibril antibody or antigen binding fragment thereof that is capable of binding to a human Aβ protofibril than in the first dose. In some embodiments, a subject having a lower second ratio relative to the first ratio is administered a different treatment for AD. A first therapeutically effective dose may be administered multiple times (e.g., biweekly or monthly for 6-18 months) before changing to a second therapeutically effective dose or dosing regimen after measuring a second Aβ42/40 ratio. In some embodiments, a first therapeutically effective dose may be administered for at least 18 months before switching to a maintenance dose. In some embodiments, a first therapeutically effective dose may be administered until a patient is amyloid negative before switching to a maintenance dose. In some embodiments, a first therapeutically effective dose may be administered until a patient is amyloid negative (e.g., as measured by amyloid or tau positron emission tomography (PET), cerebrospinal fluid level of Aβ1-42 and/or Aβ1-42/1-40 ratio, cerebrospinal fluid level of total tau, cerebrospinal fluid level of neurogranin, cerebrospinal fluid level of neurofilament light peptide (NfL), and blood biomarkers as measured in the serum or plasma (e.g. levels of Aβ1-42, the ratio of two forms of amyloid-D peptide (Aβ1-42/1-40 ratio), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and/or neurofilament light (NfL)) before switching to a maintenance dose. In some embodiments, a first therapeutically effective dose may be administered until a patient is amyloid negative, e.g., as measured by an Aβ42/40 ratio at or above 0.092-0.094 (e.g., at or above 0.092) or a florbetapir amyloid PET SUVr negativity at or below 1.17, before switching to a maintenance dose. In some embodiments, a first therapeutically effective dose may be administered until a patient is amyloid negative, e.g., as measured by an Aβ42/40 ratio above 0.092 or a florbetapir amyloid PET SUVr negativity at or below 1.17, before switching to a maintenance dose. In some embodiments, a first therapeutically effective dose comprises administering intravenously an anti-Aβ protofibril antibody or antigen binding fragment thereof that is capable of binding to a human AD protofibril at 10 mg/kg biweekly, e.g., for at least 18 months or e.g., until a patient is amyloid-negative before switching to a maintenance dose.

In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to an intravenous maintenance dose (e.g., at 10 mg/kg, e.g., biweekly, or every 4, 6, 8, 10, or 12 weeks). In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to a biweekly intravenous maintenance dose. In some embodiments, a first therapeutically effective dose comprises administering intravenously an anti-Aβ protofibril antibody or antigen binding fragment thereof that is capable of binding to a human Aβ protofibril at 10 mg/kg (e.g., administering lecanemab at 10 mg/kg), biweekly, e.g., for at least 18 months or e.g., until a patient is amyloid-negative before switching to a monthly intravenous maintenance dose. In some embodiments, a first therapeutically effective dose comprises administering intravenously an anti-Aβ protofibril antibody at 10 mg/kg biweekly, e.g., for at least 18 months or e.g., until a patient is amyloid-negative before switching to an intravenous maintenance dose every six weeks. In some embodiments, a first therapeutically effective dose comprises administering intravenously an anti-Aβ protofibril antibody or antigen binding fragment thereof that is capable of binding to a human Aβ protofibril at 10 mg/kg (e.g., administering lecanemab at 10 mg/kg), biweekly, e.g., for at least 18 months or e.g., until a patient is amyloid-negative before switching to an intravenous maintenance dose every eight weeks. In some embodiments, a first therapeutically effective dose comprises administering intravenously an anti-Aβ protofibril antibody at 10 mg/kg biweekly, e.g., for at least 18 months or e.g., until a patient is amyloid-negative before switching to an intravenous maintenance dose every two months. In some embodiments, a first therapeutically effective dose comprises administering intravenously an anti-Aβ protofibril antibody or antigen binding fragment thereof that is capable of binding to a human Aβ protofibril at 10 mg/kg (e.g., administering lecanemab at 10 mg/kg), biweekly, e.g., for at least 18 months or e.g., until a patient is amyloid-negative before switching to a quarterly intravenous maintenance dose.

In some embodiments, a first therapeutically effective dose comprises subcutaneously administering an anti-Aβ protofibril antibody at 720 mg weekly, e.g., for at least 18 months or e.g., until a patient is amyloid-negative before switching to a subcutaneous maintenance dose (e.g., at 720 mg, e.g., weekly, biweekly, or every 4, 6, 8, 10, or 12 weeks). In some embodiments, the maintenance dose is 360 mg weekly.

In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to a weekly subcutaneous maintenance dose (e.g., at a dose of 720 mg). In some embodiments, a first therapeutically effective dose comprises administering intravenously an anti-Aβ protofibril antibody or antigen binding fragment thereof that is capable of binding to a human AR protofibril at 10 mg/kg biweekly, e.g., for at least 18 months or e.g., until a patient is amyloid-negative before switching to a weekly subcutaneous maintenance dose (e.g., at a dose of 360 mg). In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to a biweekly subcutaneous maintenance dose (e.g., at a dose of 720 mg or at a dose of 360 mg). In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to a subcutaneous maintenance dose (e.g., at a dose of 720 mg or at a dose of 360 mg) every month. In some embodiments, a first therapeutically effective dose comprises administering intravenously an anti-Aβ protofibril antibody or antigen binding fragment thereof that is capable of binding to a human Aβ protofibril at 10 mg/kg (e.g., administering lecanemab at 10 mg/kg), biweekly, e.g., for at least 18 months or e.g., until a patient is amyloid-negative before switching to a subcutaneous maintenance dose (e.g., at a dose of 720 mg or at a dose of 360 mg) every six weeks. In some embodiments, a first therapeutically effective dose comprises administering intravenously an anti-Aβ protofibril antibody at 10 mg/kg biweekly, e.g., for at least 18 months or e.g., until a patient is amyloid-negative before switching to a subcutaneous maintenance dose (e.g., at a dose of 720 mg or at a dose of 360 mg) every eight weeks. In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to a subcutaneous maintenance dose (e.g., at a dose of 720 mg or at a dose of 360 mg) every two months. In some embodiments, a first therapeutically effective dose comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative before switching to a quarterly subcutaneous maintenance dose (e.g., at a dose of 720 mg or at a dose of 360 mg).

In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a weekly subcutaneous maintenance dose (e.g., at a dose of 720 mg or at a dose of 360 mg). In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a biweekly subcutaneous maintenance dose (e.g., at a dose of 720 mg). In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a weekly subcutaneous maintenance dose (e.g., a single dose of 360 mg). In some embodiments, a first therapeutically effective dose comprises subcutaneously administering an anti-Aβ protofibril antibody or antigen binding fragment thereof that is capable of binding to a human AR protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a monthly subcutaneous maintenance dose (e.g., at a dose of 720 mg). In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every six weeks. In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every eight weeks. In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every two months. In some embodiments, a first therapeutically effective dose comprises subcutaneously administering an anti-Aβ protofibril antibody or antigen binding fragment thereof that is capable of binding to a human Aβ protofibril weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a quarterly subcutaneous maintenance dose (e.g., at a dose of 720 mg).

In some embodiments, a treatment comprises administering intravenously an anti-Aβ protofibril antibody or antigen binding fragment thereof that is capable of binding to a human Aβ protofibril at 10 mg/kg biweekly, e.g., for at least 18 months. In some embodiments, a treatment comprises administering intravenously an anti-Aβ protofibril antibody or antigen binding fragment thereof that is capable of binding to a human Aβ protofibril before switching to a maintenance dose. In some embodiments, a treatment comprises administering intravenously an anti-Aβ protofibril antibody or antigen binding fragment thereof that is capable of binding to a human Aβ protofibril at 10 mg/kg biweekly, e.g., for at least 18 months before switching to a maintenance dose. In some embodiments, a subject is switched to a maintenance dose without an initial titrating step to the maintenance dose. In some embodiments, a subject is switched to a maintenance dose with at least one titrating step to the maintenance dose, e.g., the subject's dosage or frequency of administration may be reduced in multiple steps until achieving a final maintenance dosing regimen (e.g., a stepwise reduction from a subcutaneous treatment dosing regimen of 720 mg weekly to a maintenance dosing regimen of 360 mg weekly or 720 mg biweekly via intermediate dosing at intermediate amounts or time periods such as 540 mg weekly or 720 mg every 10 days). In some embodiments, a subject's maintenance dose is the same as the dose during the treatment period. In some embodiments, a subject's maintenance dose is 50% of the dose during the treatment period.

In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a weekly intravenous maintenance dose. In some embodiments, a treatment comprises administering intravenously an anti-Aβ protofibril antibody or antigen binding fragment thereof that is capable of binding to a human AR protofibril at 10 mg/kg, biweekly, e.g., for at least 18 months or e.g., until a patient is amyloid-negative, before switching to a biweekly intravenous maintenance dose. In some embodiments, a treatment comprises administering intravenously an anti-Aβ protofibril antibody or antigen binding fragment thereof that is capable of binding to a human AR protofibril at 10 mg/kg, biweekly, e.g., for at least 18 months or e.g., until a patient is amyloid-negative, before switching to a monthly intravenous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative before switching to a quarterly intravenous maintenance dose.

In some embodiments, a maintenance dose is administered subcutaneously (e.g., as a subcutaneous injection). In other embodiments, a treatment comprises subcutaneously administering an anti-Aβ protofibril antibody or antigen binding fragment thereof that is capable of binding to a human Aβ protofibril before switching to an intravenous maintenance dose. In some embodiments, a treatment comprises administering intravenously an anti-Aβ protofibril antibody or antigen binding fragment thereof that is capable of binding to a human Aβ protofibril before switching to a subcutaneous maintenance dose. In some embodiments, a treatment comprises administering intravenously an anti-Aβ protofibril antibody or antigen binding fragment thereof that is capable of binding to a human Aβ protofibril at 10 mg/kg biweekly, e.g., for at least 18 months or e.g., until a patient is amyloid-negative, before switching to a subcutaneous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a weekly subcutaneous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months e.g., until a patient is amyloid-negative, before switching to a weekly, 360 mg, subcutaneous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a weekly, 720 mg, subcutaneous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a biweekly, 720 mg, subcutaneous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a monthly, 720 mg, subcutaneous maintenance dose. In some embodiments, a treatment comprises administering intravenously an anti-Aβ protofibril antibody or antigen binding fragment thereof that is capable of binding to a human Aβ protofibril at 10 mg/kg, biweekly, e.g., for at least 18 months or e.g., until a patient is amyloid-negative, before switching to a quarterly, 720 mg, subcutaneous maintenance dose.

In some embodiments, a patient will begin treatment comprising administering intravenously an anti-Aβ protofibril antibody or antigen binding fragment thereof that is capable of binding to a human Aβ protofibril at a dose of 10 mg/kg, then switch to a treatment comprising subcutaneously administering an anti-Aβ protofibril antibody or antigen binding fragment thereof that is capable of binding to a human Aβ protofibril, e.g., at a dose of 720 mg. In some embodiments, a patient will begin treatment comprising administering intravenously an anti-Aβ protofibril antibody or antigen binding fragment thereof that is capable of binding to a human Aβ protofibril at 10 mg/kg, biweekly, then switch to a treatment comprising subcutaneously administering lecanemab weekly, e.g., at a dose of 720 mg, e.g., for a total treatment period of at least 18 months or until a patient is amyloid-negative. In some embodiments, a patient will begin treatment comprising administering intravenously an anti-Aβ protofibril antibody or antigen binding fragment thereof that is capable of binding to a human Aβ protofibril at 10 mg/kg, biweekly, then switch to a treatment comprising subcutaneously administering lecanemab weekly, e.g., at a dose of 720 mg, before switching to a weekly, 360 mg, subcutaneous maintenance dose. In some embodiments, a patient will begin treatment comprising administering intravenously an anti-Aβ protofibril antibody or antigen binding fragment thereof that is capable of binding to a human Aβ protofibril at 10 mg/kg, biweekly, then switch to a treatment comprising subcutaneously administering lecanemab weekly, e.g., at a dose of 720 mg, before switching to a monthly, 720 mg, subcutaneous maintenance dose.

In some embodiments, the maintenance dose is administered as a subcutaneous injection of the anti-Aβ protofibril antibody or antigen binding fragment thereof that is capable of binding to a human Aβ protofibril (e.g., lecanemab). In some embodiments, the maintenance dose is administered as a weekly subcutaneous injection of the subcutaneous formulation of the anti-Aβ protofibril antibody or antigen binding fragment thereof that is capable of binding to a human Aβ protofibril. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation.

In some embodiments, the anti-Aβ protofibril antibody or antigen binding fragment thereof that is capable of binding to a human Aβ protofibril is administered subcutaneously at a dose ranging from 300 mg to 800 mg, or from 400 to 1500 mg. In some embodiments, the anti-Aβ protofibril antibody or antigen binding fragment thereof that is capable of binding to a human Aβ protofibril is administered subcutaneously at a dose of 300 mg to 400 mg. In some embodiments, the anti-Aβ protofibril antibody or antigen binding fragment thereof that is capable of binding to a human Aβ protofibril is administered subcutaneously at a dose of 400 mg to 500 mg. In some embodiments, the anti-Aβ protofibril antibody or antigen binding fragment thereof that is capable of binding to a human AR protofibril is administered subcutaneously at a dose of 400 mg to 450 mg. In some embodiments, the anti-Aβ protofibril antibody or antigen binding fragment thereof that is capable of binding to a human Aβ protofibril is administered subcutaneously at a dose of 450 mg to 500 mg. In some embodiments, the anti-Aβ protofibril antibody or antigen binding fragment thereof that is capable of binding to a human Aβ protofibril is administered subcutaneously at a dose of 500 mg to 600 mg. In some embodiments, the anti-Aβ protofibril antibody or antigen binding fragment thereof that is capable of binding to a human Aβ protofibril is administered subcutaneously at a dose of 500 mg to 550 mg. In some embodiments, the anti-Aβ protofibril antibody or antigen binding fragment thereof that is capable of binding to a human Aβ protofibril is administered subcutaneously at a dose of 550 mg to 600 mg. In some embodiments, the anti-Aβ protofibril antibody or antigen binding fragment thereof that is capable of binding to a human Aβ protofibril is administered subcutaneously at a dose of 600 mg to 700 mg. In some embodiments, the anti-Aβ protofibril antibody or antigen binding fragment thereof that is capable of binding to a human Aβ protofibril is administered subcutaneously at a dose of 600 mg to 650 mg. In some embodiments, the anti-Aβ protofibril antibody or antigen binding fragment thereof that is capable of binding to a human Aβ protofibril is administered subcutaneously at a dose of 650 mg to 700 mg. In some embodiments, the anti-Aβ protofibril antibody or antigen binding fragment thereof that is capable of binding to a human Aβ protofibril is administered subcutaneously at a dose of 700 mg to 800 mg. In some embodiments, the anti-Aβ protofibril antibody or antigen binding fragment thereof that is capable of binding to a human Aβ protofibril is administered subcutaneously at a dose of 700 mg to 750 mg. In some embodiments, the anti-Aβ protofibril antibody or antigen binding fragment thereof that is capable of binding to a human Aβ protofibril is administered subcutaneously at a dose of 750 mg to 800 mg. In some embodiments, the anti-Aβ protofibril antibody or antigen binding fragment thereof that is capable of binding to a human Aβ protofibril is administered subcutaneously at a dose of 300 mg, 310 mg, 320 mg, 330 mg, 340 mg, 350 mg, 360 mg, 370 mg, 380 mg, or 390 mg. In some embodiments, the anti-Aβ protofibril antibody or antigen binding fragment thereof that is capable of binding to a human Aβ protofibril is administered subcutaneously at a dose of 400 mg, 410 mg, 420 mg, 430 mg, 440 mg, 450 mg, 460 mg, 470 mg, 480 mg, or 490 mg. In some embodiments, the anti-Aβ protofibril antibody or antigen binding fragment thereof that is capable of binding to a human Aβ protofibril is administered subcutaneously at a dose of 500 mg, 510 mg, 520 mg, 530 mg, 540 mg, 550 mg, 560 mg, 570 mg, 580 mg, or 590 mg. In some embodiments, the anti-Aβ protofibril antibody or antigen binding fragment thereof that is capable of binding to a human Aβ protofibril is administered subcutaneously at a dose of 600 mg, 610 mg, 620 mg, 630 mg, 640 mg, 650 mg, 660 mg, 670 mg, 680 mg, or 690 mg. In some embodiments, the anti-Aβ protofibril antibody or antigen binding fragment thereof that is capable of binding to a human Aβ protofibril is administered subcutaneously at a dose of 700 mg, 710 mg, 720 mg, 730 mg, 740 mg, 750 mg, 760 mg, 770 mg, 780 mg, or 790 mg. In some embodiments, the anti-Aβ protofibril antibody or antigen binding fragment thereof that is capable of binding to a human Aβ protofibril is administered subcutaneously at a dose of 440 mg. In some embodiments, the anti-Aβ protofibril antibody or antigen binding fragment thereof that is capable of binding to a human Aβ protofibril is administered subcutaneously at a dose of 580 mg. In some embodiments, the anti-Aβ protofibril antibody or antigen binding fragment thereof that is capable of binding to a human Aβ protofibril is administered subcutaneously at a dose of 720 mg.

In some embodiments, the anti-Aβ protofibril antibody or antigen binding fragment thereof that is capable of binding to a human Aβ protofibril is administered subcutaneously in a dose ranging from 800 mg to 1600 mg. In some embodiments, the anti-Aβ protofibril antibody or antigen binding fragment thereof that is capable of binding to a human Aβ protofibril is administered subcutaneously in a dose of 800 mg to 1000 mg. In some embodiments, the anti-Aβ protofibril antibody or antigen binding fragment thereof that is capable of binding to a human Aβ protofibril is administered subcutaneously at a dose of 800 mg to 900 mg. In some embodiments, the anti-Aβ protofibril antibody or antigen binding fragment thereof that is capable of binding to a human Aβ protofibril is administered subcutaneously at a dose of 900 mg to 1000 mg. In some embodiments, the anti-Aβ protofibril antibody or antigen binding fragment thereof that is capable of binding to a human Aβ protofibril is administered subcutaneously at a dose of 1000 mg to 1200 mg. In some embodiments, the anti-Aβ protofibril antibody or antigen binding fragment thereof that is capable of binding to a human Aβ protofibril is administered subcutaneously at a dose of 1000 mg to 1100 mg. In some embodiments, the anti-Aβ protofibril antibody or antigen binding fragment thereof that is capable of binding to a human Aβ protofibril is administered subcutaneously at a dose of 1100 mg to 1200 mg. In some embodiments, the anti-Aβ protofibril antibody or antigen binding fragment thereof that is capable of binding to a human Aβ protofibril is administered subcutaneously at a dose of 1200 mg to 1400 mg. In some embodiments, the anti-Aβ protofibril antibody or antigen binding fragment thereof that is capable of binding to a human Aβ protofibril is administered subcutaneously at a dose of 1200 mg to 1300 mg. In some embodiments, the anti-Aβ protofibril antibody or antigen binding fragment thereof that is capable of binding to a human Aβ protofibril is administered subcutaneously at a dose of 1300 mg to 1400 mg. In some embodiments, the anti-Aβ protofibril antibody or antigen binding fragment thereof that is capable of binding to a human Aβ protofibril is administered subcutaneously at a dose of 1400 mg to 1600 mg. In some embodiments, the anti-Aβ protofibril antibody or antigen binding fragment thereof that is capable of binding to a human Aβ protofibril is administered subcutaneously at a dose of 1400 mg to 1500 mg. In some embodiments, the anti-Aβ protofibril antibody or antigen binding fragment thereof that is capable of binding to a human Aβ protofibril is administered subcutaneously at a dose of 1500 mg to 1600 mg. In some embodiments, the anti-Aβ protofibril antibody or antigen binding fragment thereof that is capable of binding to a human Aβ protofibril is administered subcutaneously at a dose of 800 mg, 820 mg, 840 mg, 860 mg, 880 mg, 900 mg, 920 mg, 940 mg, 960 mg, or 960 mg. In some embodiments, the anti-Aβ protofibril antibody or antigen binding fragment thereof that is capable of binding to a human Aβ protofibril is administered subcutaneously at a dose of 1000 mg, 1020 mg, 1040 mg, 1060 mg, 1080 mg, 1100 mg, 1120 mg, 1140 mg, 1160 mg, or 1180 mg. In some embodiments, the anti-Aβ protofibril antibody or antigen binding fragment thereof that is capable of binding to a human Aβ protofibril is administered subcutaneously at a dose of 1200 mg, 1220 mg, 1240 mg, 1260 mg, 1280 mg, 1300 mg, 1320 mg, 1340 mg, 1360 mg, or 1380 mg. In some embodiments, the anti-Aβ protofibril antibody or antigen binding fragment thereof that is capable of binding to a human Aβ protofibril is administered subcutaneously at a dose of 1400 mg, 1400 mg, 1440 mg, 1460 mg, 1480 mg, 1500 mg, 1520 mg, 1540 mg, 1560 mg, or 1580 mg. In some embodiments, the anti-Aβ protofibril antibody or antigen binding fragment thereof that is capable of binding to a human Aβ protofibril is administered subcutaneously at a dose of 880 mg. In some embodiments, the anti-Aβ protofibril antibody or antigen binding fragment thereof that is capable of binding to a human Aβ protofibril is administered subcutaneously at a dose of 1160 mg. In some embodiments, the anti-Aβ protofibril antibody or antigen binding fragment thereof that is capable of binding to a human Aβ protofibril is administered subcutaneously at a dose of 1440 mg.

Further exemplary subcutaneous doses, dosing frequencies, maintenance doses, and formulations for subcutaneous administration that may be used in the methods discussed herein are disclosed in International Application No. PCT/US2022/041926, the contents of which are fully incorporated herein by reference.

In some embodiments, the anti-Aβ protofibril antibody or antigen binding fragment thereof used in the methods disclosed herein is delivered by autoinjector. In some embodiments, the autoinjector delivers the anti-Aβ protofibril antibody or antigen binding fragment thereof subcutaneously. In some embodiments, the autoinjector delivers an injection volume of 1.1 mL. In some embodiments, the autoinjector delivers an injection volume of 1.4 mL. In some embodiments, the autoinjector delivers an injection volume of 1.8 mL. In some embodiments, the autoinjector delivers about 400-800 mg, e.g., 360 mg of an anti-Aβ protofibril antibody or antigen binding fragment thereof subcutaneously.

### EXAMPLES

The present disclosure is further illustrated by the following examples that should not be construed as limiting. The contents of all references, patents, and published patent applications cited throughout this application, as well as the figures, are incorporated herein by reference in their entirety for all purposes.

### Example 1: Treatment of Alzheimer's Disease with Lecanemab Administered in Conjunction with E2814

### 1.1 Dose Selection in Double-Blind Period

### 1.1.1 E2814

1500 mg of E2814, an anti-tau antibody or antigen binding fragment thereof, is administered intravenously (IV), every 4 weeks. A higher dose (3000 mg or 4500 mg) study may be considered in a future protocol amendment, based on safety, PK, and TE data of higher doses.

### 1.1.2 Lecanemab

Lecanemab treatment will be administered at 10 mg/kg dose via IV infusion Q2W.

### 1.2 Drug-Specific Study Design

This is a placebo-controlled, double-blind phase II/III study to evaluate the therapeutic effects of E2814, when administered alone or concurrently with lecanemab, on safety, tolerability, biomarker, cognitive, and clinical efficacy.

Inclusion criteria include:
Individuals between 18-80 years of age;
Individuals who know they have an Alzheimer's disease-causing mutation;
Are within −10 to +10 years of the predicted or actual age of cognitive symptom onset;
Cognitively normal or with mild cognitive impairment or mild dementia, Clinical Dementia Rating (CDR) of 0-1 (inclusive);
Fluency in approved trial language and evidence of adequate premorbid intellectual functioning;
Able to undergo Magnetic Resonance Imaging (MRI), Lumbar Puncture (LP), Positron Emission Tomography (PET), and complete all study related testing and evaluations.
For women of childbearing potential, if partner is not sterilized, participant must agree to use effective contraceptive measures (hormonal contraception, intra-uterine device, sexual abstinence, barrier method with spermicide);
Adequate visual and auditory abilities to perform all aspects of the cognitive and functional assessments;
Has a Study Partner who in the investigator's judgment is able to provide accurate information as to the subject's cognitive and functional abilities, who agrees to provide information at the study visits which require informant input for scale completion.

### Exclusion Criteria Include:

Significant neurologic disease (other than AD) or psychiatric disease that may currently or during the course of the study affect cognition or participant's ability to complete the study;
At high risk for suicide, e.g., significant suicidal ideation or attempt within last 12 months—current stable mild depression or current use of antidepressant medications is not exclusionary;
History or presence of brain MRI scans indicative of any other significant abnormality;
Substance or alcohol use disorder currently or within the past 1 year;
Presence of pacemakers, aneurysm clips, artificial heart valves, ear implants, or foreign metal objects in the eyes, skin or body which would preclude MRI scan;
History or presence of clinically significant cardiovascular disease, hepatic/renal disorders, infectious disease or immune disorder, or metabolic/endocrine disorders;
On anticoagulants other than low dose (≤325 mg) aspirin;
Have been exposed to a monoclonal antibody targeting beta amyloid peptide within the past six months;
History of cancer within the last 5 years, except basal cell carcinoma, non-squamous skin carcinoma, prostate cancer or carcinoma in situ with no significant progression over the past 2 years;
Positive urine or serum pregnancy test or plans or desires to become pregnant during the course of the trial;
Subjects unable to complete all study related testing, including implanted metal that cannot be removed for MRI scanning, required anticoagulation and pregnancy.

Approximately 168 participants will be enrolled into the E2814 blinded study drug arm with 84 participants in each of two cohorts. All 168 participants within −10 to +10 estimated years from symptom onset (EYO) will be assigned into 2 cohorts based on their baseline (Week 0) CDR score as follows:

Symptomatic Population (Cohort 1): A minimum of 84 participants with CDR=0.5-1 (symptomatic with mild cognitive impairment)

Asymptomatic Population (Cohort 2): A minimum of 84 participants with CDR=0 (cognitively normal)

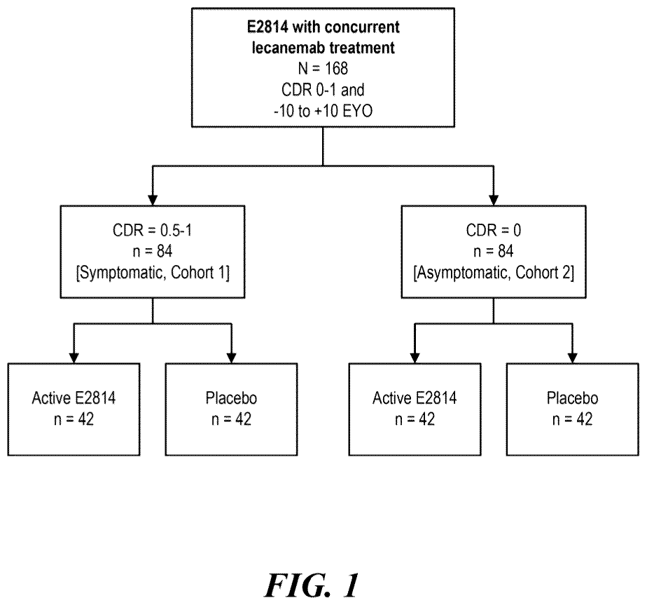

Participants will be randomized 1:1 active E2814 to placebo (FIG. 1) via a minimization algorithm including the following factors at the time of randomization:
1. Clinical Dementia Rating-Sum of Boxes (CDR-SB; 0, 0.5 to 1, 1.5 to 3, >3)
2. Estimated years from symptom onset (EYO) (−10 to −5, −4 to −1, 0 to 4, 5 to 10)
3. Gene type (APP, PSEN1, PSEN2)
4. Years of education (<12, 12, 13 to 16, >16)
5. Age (18 to 40, 41 to 55, 56 to 80 years)
6. Presence of APOE4 allele (APOE4 positive, APOE4 negative)
7. Region (US/Australia/Canada, Europe, Rest of World)
8. Study site, and
9. Sex (Male, Female).

The minimization randomization algorithm will be employed in order to achieve two goals: 1) to balance prognostic factors that affect or non-prognostic factors that may affect clinical and cognitive progression (these factors include baseline CDR-SB, EYO, age at randomization, years of education, and region), and 2) to balance factors that may affect adverse events or interfere with drug effects (these factors include genotype, APOE4 allele, and sex).

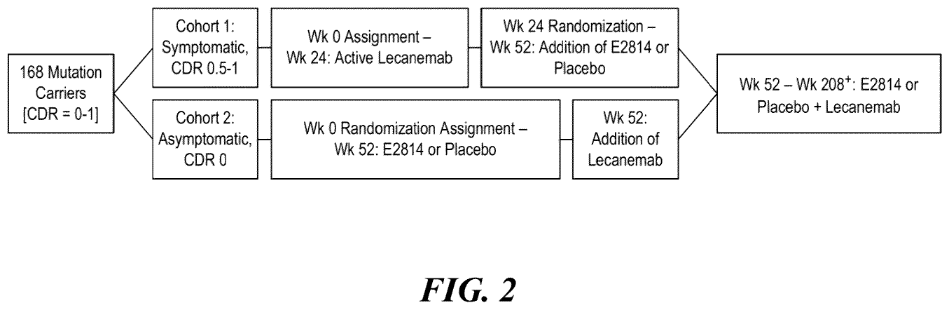

E2814 or placebo will be concurrently administered with open-label lecanemab as part of the E2814 blinded study drug arm. E2814 or placebo will be administered intravenously every 4 weeks (Q4W) at a dose of 1500 mg and lecanemab administered intravenously every two weeks (Q2W) at 10 mg/kg with each cohort initiating treatment of each at different time points, as specified in FIG. 2. Participants will continue to receive treatment until all concurrently enrolled participants receive a minimum of 4 years (210 weeks) treatment or is withdrawn (common close). Each cohort will initiate treatment of E2814 or placebo, and lecanemab, for concurrent administration as follows:

### Symptomatic Population (Cohort 1)

At Week 0, participants will receive open-label lecanemab 10 mg/kg administered intravenously every two weeks (Q2W) through the full treatment period.

At Week 24, participants will be randomized in a 1:1 ratio to receive 1500 mg E2814 or placebo intravenously Q4W in a blinded fashion the remainder of their treatment period.

### Asymptomatic Population (Cohort 2)

At Week 0, participants will be randomized in a 1:1 ratio to receive 1500 mg E2814 or placebo intravenously Q4W in a blinded fashion for the full treatment period.

At Week 52, all participants will initiate open-label lecanemab 10 mg/kg administered intravenously Q2W for the remainder of their treatment period.

By staggering the administration of each drug in this manner, it is possible to evaluate the effects of lecanemab and E2814 alone before evaluation of the drugs together.

### 1.3 Rationale for Biomarker Objectives and Endpoints

Based on the tau pathophysiology evolvement through distinct phases in DIAD, the primary study endpoint for the symptomatic population (Cohort 1) is tau PET, and the key secondary study endpoint for asymptomatic population (Cohort 2) is cerebrospinal fluid (CSF) phosphorylated tau (ptau217)/total tau ratio (p-tau217/total tau ratio).

There was a dose-related decrease in free MTBR-tau following single E2814 infusions of 3, 10 and 30 mg/kg, substantiating that E2814 is able to lower MTBR-tau levels in CSF. E2814-bound and free MTBR-tau were used to estimate target engagement (TE) as the fraction (%) of bound MTBR-tau relative to total MTBR-tau (free plus bound). These preliminary data suggest there is a dose-related increase in TE of approximately 26%, 45%, and 60% (3, 10 and 30 mg/kg, respectively) with sustained TE up to Day 29.

### 1.4 Primary Study Objective and Endpoint

The primary study endpoint is to determine whether E2814 is superior to placebo, when each is concurrently administered with lecanemab, in the change from Week 24 to Week 104 (interim analysis) and Week 208 (final analysis) in tau spread as measured by tau PET in the symptomatic population (Cohort 1).

### 1.5 Secondary Study Endpoints

### 1.5.1 Key Secondary Endpoints

The key secondary endpoints are as follows:

Symptomatic Population (Cohort 1): To determine whether E2814 is superior to placebo, when each is concurrently administered with lecanemab, in change from Week 24 to Week 208 in Clinical Dementia Rating Scale Sum of Boxes (CDR-SB)

Asymptomatic Population (Cohort 2): To determine whether E2814 is superior to placebo, when each is administered alone and then concurrently with lecanemab, in change from Week 0 to Week 104 (interim analysis) and Week 208 (final analysis) in cerebrospinal fluid (CSF) phosphorylated tau (p-tau217)/total tau

### 1.5.2 Additional Secondary Endpoints

Additional secondary endpoints for the individual or combined population cohorts are as follows:

Symptomatic Population (Cohort 1): 1) to determine whether E2814 is superior to placebo, when each is concurrently administered with lecanemab, in change from Week 24 to Week 104 and Week 208 in a cognitive composite score, 2) to assess the effect of lecanemab, when administered alone, in change on amyloid PET from Week 0 to Week 24, 3) to determine whether lecanemab, when administered alone or with placebo, is superior to the external control in change on CDR-SB from Week 0 to Week 208, 4) to assess safety and tolerability including assessment of immunogenicity (production of anti-lecanemab antibodies) of lecanemab, when administered alone for 24 weeks, and 5) to determine whether E2814 is superior to placebo, when each is concurrently administered with lecanemab, in change from Week 24 to Week 104 and Week 208 in CSF neurofilament light (NFL).

Asymptomatic Population (Cohort 2): 1) to evaluate whether E2814 is superior to placebo, when each is administered alone, in change from Week 0 to Week 52 in CSF p-tau217/total tau, 2) to determine whether E2814 is superior to placebo, when each is concurrently administered with lecanemab, in change from Week 52 to Week 104 (interim analysis) and Week 208 (final analysis) in CSF p-tau217)/total tau, 3) to assess the safety and tolerability including assessment of immunogenicity (production of anti-E2814 antibodies) of E2814 when administered alone for 52 weeks, 4) to determine whether E2814 is superior to placebo, when each is concurrently administered with lecanemab, in change from Week 52 to Week 104 and Week 208 in CSF neurofilament light (NFL).

### 1.6 Exploratory Endpoints

The following are exploratory endpoints for the respective population cohorts in the E2814 arm of the study:

### Symptomatic Population (Cohort 1):

To evaluate the effects of E2814 compared with placebo, when each is concurrently administered with lecanemab, in change from Week 24 to Week 104 and Week 208 in reducing CSF p-tau217/total tau
To evaluate the effects of lecanemab, when administered alone, in change from Week 0 to Week 24 in tau PET, CSF and blood biomarkers (amyloid beta [Aβ]42, Aβ40, p-tau, total tau, NFL)

### Asymptomatic Population (Cohort 2):

To evaluate whether E2814 is superior to placebo, when each is concurrently administered with lecanemab, in change from Week 52 to Week 104 and Week 208 in CSF p-tau217/total tau
To evaluate the effects of E2814 compared with placebo, when each is concurrently administered with lecanemab, in change from Week 52 to Week 104 and Week 208 in a cognitive composite score
To evaluate the effects of E2814 compared with placebo, when each is concurrently administered with lecanemab, in change from Week 52 to Week 104 and Week 208 in reducing brain tau spread as measured by tau PET
To evaluate whether E2814 is superior to placebo, when administered alone, in change from Week 0 to Week 52 in tau PET, CSF biomarkers (NFL and MTBR-tau) and blood biomarkers (p-tau217/total tau and NFL)

### Symptomatic Population (Cohort 1) and Asymptomatic Population (Cohort 2):

To evaluate the effects of E2814 compared with placebo, when each is concurrently administered with lecanemab, in change from Week 24 to Week 104 (Cohort 1), and from Week 52 to Week 104 and Week 208 (Cohort 2) in CDR-SB
To evaluate the effects of E2814 compared with placebo, when each is concurrently administered with lecanemab, in change from Week 24 to Week 104 and Week 208 (Cohort 1) and from Week 52 to Week 104 and Week 208 (Cohort 2) in CSF microtubule binding region (MTBR)-tau
To evaluate whether E2814 is superior to placebo, when each is concurrently administered with lecanemab, in change from Week 24 to Week 104 and Week 208 (Cohort 1) and from Week 52 to Week 104 and Week 208 (Cohort 2) in plasma NFL
To evaluate whether E2814 is superior to placebo, when each is concurrently administered with lecanemab, in change from Week 24 to Week 104 and Week 208 (Cohort 1) and from Week 52 to Week 104 and Week 208 (Cohort 2) in CSF and blood biomarkers (other than those already listed above) of neurodegeneration, neuroinflammation, amyloid, and tau
To evaluate the effects of E2814 compared with placebo, when each is concurrently administered with lecanemab, in change from Week 24 to Week 208 (Cohort 1) and from Week 52 to Week 208 (Cohort 2) in cognitive and functional endpoints to include the following:
   Geriatric Depression Scale (GDS)
   Neuropsychiatric Inventory Questionnaire (NPI-Q)
   Functional Assessment Scale (FAS)
   Mini-Mental State Examination (MMSE)
   DIAN Memory Complaint Questionnaire (MAC-Q)
   Buschke and Grober Free and Cued Selective Reminding Test-Immediate Recall (FCSRT-IR)
   Wechsler Memory Scale Revised (WMS-R) Logical Memory/Paragraph Memory (Immediate & Delayed Recall), Alternate Paragraph for Logical Memory I & II—Version A (Immediate and Delayed) and Alternate Paragraph for Logical Memory I & II—Version B (Immediate and Delayed)
   Category Fluency (Animals)
   Weschler Adult Intelligence Scale-Revised (WAIS-R) Digit-Symbol Substitution Test
   Trailmaking Test Parts A & B
   Weschler Memory Scale-Revised (WMS-R) Digit Spatial Span Forward and Backward)
   Ambulatory Research in Cognition (ARC) smartphone-based cognitive assessments (Grids, Prices, Symbols)
To evaluate the effects of E2814 compared with placebo, when each is concurrently administered with lecanemab, in change from Week 24 to Week 104 and Week 208 (Cohort 1) and from Week 52 to Week 104 and Week 208 (Cohort 2) in imaging measures at to include the following:
   Amyloid load based on (11)C-labelled Pittsburgh Compound-B ([11C]PiB) PET
   Fluorodeoxyglucose (FDG)-PET metabolism in specific regions of interest (eg, precuneus)
   Rate of brain atrophy as measured by cortical thickness of regions of interest, including whole brain volume and ventricular volume (volumetric magnetic resonance imaging [vMRI])
   Functional connectivity MRI (fc-MRI)
   Parameters in Diffusion Tensor Imaging (DTI) MRI, including diffusion basis spectrum imaging (DBSI)
   Blood flow measures by Arterial Spin Labelling (ASL) MRI
   Assessment of clinical MRI features, such as microhemorrhages (MCH), white matter hyperintensities (WMH), cerebral infarctions, and amyloid related imaging abnormalities (ARIA) using conventional MRI sequences
To assess the population pharmacokinetics (PK) of E2814 in plasma and serum
To assess the population PK of lecanemab in serum
To explore PK (E2814/lecanemab exposure)-pharmacodynamic (PD) (CSF, blood, and imaging biomarkers) relationship
To collect pharmacogenomics (PG) samples for future analysis

### 1.7 Safety Objectives and Endpoints

This study will assess safety and tolerability of treatment with E2814 and lecanemab in individuals with DIAD. The safety endpoints include AEs, clinical laboratory results, vital signs, ECGs, CSSRS, and physical and neurological examinations.

Assessment of immunogenicity (production of anti-E2814 antibodies and anti-lecanemab antibodies) of concurrent administration of E2814 and lecanemab.

Safety measures related to plasma fibrinogen binding include monitoring of functional fibrinogen using the Clauss method.

Safety MRIs to monitor for ARIA will also be evaluated.

### 1.8 Drug-specific Tests

### 1.8.1 Pharmacokinetic Assessments

### 1.8.1.1 Blood PK for E2814 and Lecanemab

Blood samples for E2814 PK assessments (plasma and serum) and lecanemab (serum) will be collected pre-dose, immediately prior to the start of any drug administration at that visit; and post-dose at any time after the 30 minutes following completion of all drug administration at that visit. Blood will be collected in year one based on the assigned cohort as outlined below, and approximately every 26 weeks (approximately 6 months) through year 4, annually thereafter and at the safety follow-up visit. Samples should also be collected should a participant terminate early.

TABLE 1
E2814 and Lecanemab Blood PK-Cohort-Specific Collection Schedule

| | Symptomatic Cohort 1 | | Asymptomatic Cohort 2 | |
|---|---|---|---|---|
| | E2814 PK | | E2814 PK | |
| | (plasma and | Lecanemab | (plasma and | Lecanemab |
| VISIT | serum) | PK (serum) | serum) | PK (serum) |
| Baseline, Week 0 | | X | X | |
| (V2) | | | | |
| Week 12 (V5) | | X | | |
| Week 24 (V8) | X | X | X | |
| Week 52 (V15) | X | X | X | X |
| Week 76 (V21) | X | X | X | X |
| Week 104 (V28) | X | X | X | X |
| Week 128 (V34) | X | X | X | X |
| Week 156 (V41) | X | X | X | X |
| Week 180 (V47) | X | X | X | X |
| Week 208 (V54) | X | X | X | X |
| Week 260 (V67) | X | X | X | X |
| Week 312 (V80) | X | X | X | X |
| Week 364 (V93) | X | X | X | X |
| Early Termination | X | X | X | X |
| Safety Follow-Up | X | X | X | X |

Serum and plasma concentrations of E2814 will be measured by validated electrochemiluminescence (ECL) assay methods.

### 1.8.1.2 E2814 and Lecanemab Cerebrospinal Fluid (CSF) PK—Cohort-Specific Collection Schedule

CSF samples for E2814 and Lecanemab PK assessments will be collected pre-dose via lumbar puncture (LP) at the below-specified visits based on the assigned cohort.

TABLE 2
E2814 and Lecanemab CSF PK-Cohort-Specific Collection Schedule

| | Symptomatic Cohort 1 | | Asymptomatic Cohort 2 | |
|---|---|---|---|---|
| | E2814 CSF | Lecanemab | E2814 CSF | Lecanemab |
| VISIT | PK | CSF PK | PK | CSF PK |
| Baseline, Week 0 | | X | X | |
| (V2) | | | | |
| Week 24 (V8) | X | X | | |
| Week 52 (V15) | X | X | X | X |
| Week 104 (V28) | X | X | X | X |
| Week 208 (V54) | X | X | X | X |
| Week 260 (V67) | X | X | X | X |
| Early Termination | X | X | X | X |

CSF concentrations of E2814 will be measured by validated electrochemiluminescence (ECL) assay methods. CSF concentrations of lecanemab will be measured by validated immunoprecipitation—liquid chromatography—tandem mass spectrometry (IP/LC-MS/MS) methods.

### 1.8.2 Immunogenicity Assessments

Blood samples for immunogenicity will be collected immediately prior to LP at annual visits requiring LP, and prior to drug administration at all other visits. Immunogenicity will be assessed by measuring serum for the presence of anti-E2814 and anti-lecanemab antibodies in year one based on the assigned cohort as outlined below, approximately every 26 weeks (approximately 6 months) through year 4, and in the event of early termination (ET).

TABLE 3
Anti-E2814 and Anti-Lecanemab Serum Antibody Cohort-Specific
Collection Schedule

| | Symptomatic Cohort 1 | | Asymptomatic Cohort 2 | |
|---|---|---|---|---|
| | | Anti- | | Anti- |
| | Anti-E2814 | Lecanemab | Anti-E2814 | Lecanemab |
| | Antibodies | Antibodies | Antibodies | Antibodies |
| VISIT | (serum) | (serum) | (serum) | (serum) |
| Baseline, Week 0 | | X | X | |
| (V2) | | | | |
| Week 12 (V5) | | X | | |
| Week 24 (V8) | X | X | X | |
| Week 52 (V15) | X | X | X | X |
| Week 76 (V21) | X | X | X | X |
| Week 104 (V28) | X | X | X | X |
| Week 128 (V34) | X | X | X | X |
| Week 156 (V41) | X | X | X | X |
| Week 180 (V47) | X | X | X | X |
| Week 208 (V54) | X | X | X | X |
| Week 260 (V67) | X | X | X | X |
| Week 312 (V80) | X | X | X | X |
| Week 364 (V93) | X | X | X | X |
| Early Termination | X | X | X | X |
| Safety Follow-Up | X | X | X | X |

For participants with significant immunogenicity at their last collected assessment (Week 208 or ET visit), anti-E2814 antibodies or anti-lecanemab antibodies will continue to be assessed every 24 weeks until resolution, when/where possible. In addition, clinical measures such as CRP to monitor for inflammation that may be associated with immunogenicity may be performed.

Serum anti-E2814 antibodies will be measured by appropriately validated ECL assay methods. Serum anti-lecanemab antibody will be measured using a validated Meso Scale Discovery® (MSD) bridging assay

### 1.8.3 Pharmacodynamic and Biomarker Assessments

Blood samples for plasma pharmacodynamic (PD) biomarkers will be collected immediately prior to LP at annual visits requiring LP, and prior to drug administration at all other visits. Samples will be collected on the same schedule for both cohorts as outlined below approximately every 12 weeks in year 1, approximately every 26 weeks (approximately every 6 months) up to year 4, and annually thereafter including the safety follow-up visit.
Year 1: Week 0 (V2), Week 12 (V5), Week 24 (V8), Week 36 (V11), Week 52 (V15)
Year 2: Week 76 (V21) and Week 104 (V28)
Year 3: Week 128 (V34) and Week 156 (V41)
Year 4: Week 180 (V47) and Week 208 (V54)
Year 5: Week 260 (V67), if applicable
Year 6: Week 312 (V80), if applicable
Year 7: Week 364 (V93), if applicable
Safety Follow-Up
Early Termination, if applicable

CSF samples for PD biomarkers will be collected pre-dose via lumbar puncture at the following visits:
Week 0 (V2), Baseline
Week 24 (V8)—Cohort 1 only
Week 52 (V15)
Week 104 (V28)
Week 208 (V54) or ET
Week 260 (V67)
Early Termination, if terminating prior to Week 260 (V67)

CSF and blood concentrations of AD-related biomarkers (including but not limited to Aβ[1-42], neurogranin, neurofilament light chain [NFL], MTBR-tau [bound, free and total], total tau [t-tau], and phosphorylated tau [p-tau]) will be measured.

### 1.8.4 Genomic DNA Blood Sample for Banking

Pharmacogenomic and biomarker samples obtained from participants of this study may be analyzed by global proteomic, metabolomic, or lipidomic and single or multiplex assays in an effort to identify predictive biomarkers for PK and PD. In addition, biomarkers identified in other clinical studies may also be assessed in samples collected from participants enrolled in this study.

### Example 2: A Treatment of Alzheimer's Disease with Lecanemab Administered Subcutaneously in Conjunction with E2814

### Dose Selection in Double-Blind Period

E2814: 3000 mg of E2814, an anti-tau antibody or antigen binding fragment thereof, is administered intravenously (IV), every 4 weeks. A higher dose (4500 mg) study may be considered in a future protocol amendment, based on safety, PK, and TE data of higher doses

Lecanemab: Lecanemab treatment will be administered subcutaneously using two autoinjectors that deliver 360 mg of lecanemab each for a total dose of 720 mg lecanemab. Patients will receive the two 360 mg doses weekly or biweekly.

Symptomatic Population (Cohort 1): At Week 0, participants will receive 720 mg open-label lecanemab administered subcutaneously using two consecutive 360 mg doses delivered using autoinjectors through the full treatment period.

At Week 24, participants will be randomized in a 1:1 ratio to receive 3000 mg E2814 or placebo intravenously Q4W in a blinded fashion the remainder of their treatment period.

Asymptomatic Population (Cohort 2): At Week 0, participants will be randomized in a 1:1 ratio to receive 3000 mg E2814 or placebo intravenously Q4W in a blinded fashion for the full treatment period.

At Week 52, all participants will initiate receive 720 mg open-label lecanemab administered subcutaneously using two consecutive 360 mg doses delivered using autoinjectors through the remainder of their treatment period.

### EMBODIMENT

### Embodiment 1: Lecanemab Background Anti-Amyloid Therapy in Next-Generation Clinical Trial (Tau NexGen Trial) Evaluating Investigational Therapy Targeting Tau for Dominantly Inherited Alzheimer's Disease

People who have this genetic mutation of DIAD are known to develop AD and will likely develop symptoms at around the same age their affected parents did, often in their 50s, 40s or even 30s. The purpose of the Tau NexGen trial is to assess the safety, tolerability, biomarker and cognitive efficacy of investigational therapies in people who have an Alzheimer's disease-causing gene mutation. The trial will evaluate if treatment with the investigational drug slows the rate of progression of cognitive impairment and improves disease-related biomarkers.

In the amended Tau NexGen trial, symptomatic participants will be administered Lecanemab for six months before being randomly assigned to also receive E2814 or a placebo. Since amyloid plaques accumulate before tau tangles in AD, this trial design allows researchers to assess whether amyloid removal clears the way for the anti-tau drug to function most effectively. Pre-symptomatic participants will be randomly assigned to receive the anti-tau drug E2814 or a placebo for a year before beginning Lecanemab administration. Staggering the drugs in this way will allow evaluation of the effects of the anti-tau drug alone before assessing the effects of the two drugs together. Lecanemab will be administered at a 10 mg/kg dose biweekly, which does not require titration dosing and has a 9.9% incidence rate of amyloid-related image abnormalities, of which less than 2% had symptoms. The primary endpoint is a slowing of tau accumulation in the brain in symptomatic participants (as an effect of adding E2814) as seen on PET brain scans. A secondary endpoint will be the effect on levels of a specific kind of tau—phosphorylated tau 217—in the cerebrospinal fluid (CSF) of pre-symptomatic participants (effect of E2814 in isolation or in combination with Lecanemab). If these primary and secondary endpoints are achieved in the analysis two years after the start of trial, the trial will be extended for another two years to assess whether the drug slows cognitive decline and has further effects on tau pathology.

### Selected Sequences

TABLE 11
Amino acid sequences of mAb CDRs

| | | SEQ | |
|---|---|---|---|
| mAb | CDR | ID NO | Amino acid sequence |
| Lecanemab | HCDR1 | 1 | SFGMH |
| Heavy Chain | HCDR2 | 2 | YISSGSSTIYYGDTVKG |
| (Kabat) | HCDR3 | 3 | EGGYYYGRSYYTMDY |
| Lecanemab | LCDR1 | 4 | RSSQSIVHSNGNTYLE |
| Light Chain | LCDR2 | 5 | KVSNRFS |
| (Kabat) | LCDR3 | 6 | FQGSHVPPT |
| E2814 | HCDR1 | 7 | TYWIT |
| Heavy Chain | HCDR2 | 8 | DIYPGSSISNYNEKFKS |
| (Kabat) | HCDR3 | 9 | EDGYDAWFAY |
| E2814 Light | LCDR1 | 10 | RSSQSILHSNGNTYLE |
| Chain | LCDR2 | 11 | KVSNRFS |
| (Kabat) | LCDR3 | 12 | FQGSHVPFT |
| E2814 | HCDR1 | 24 | GYTFTTYW |
| Heavy Chain | HCDR2 | 25 | IYPGSSIS |
| (IMGT) | HCDR3 | 26 | AREDGYDAWFAY |
| E2814 Light | LCDR1 | 27 | QSILHSNGNTY |
| Chain | LCDR2 | 28 | KVS |
| (IMGT) | LCDR3 | 29 | FQGSHVPFT |

TABLE 12
Amino acid sequences of mAb variable regions

| | SEQ | |
|---|---|---|
| mAb | ID NO | Amino acid sequence |
| Lecanemab | 13 | EVQLVESGGGLVQPGGSLRLSCSASGF |
| heavy chain | | TFSSFGMHWVRQAPGKGLEWVAYISS |
| variable | | GSSTIYYGDTVKGRFTISRDNAKNSLFL |
| domain | | QMSSLRAEDTAVYYCAREGGYYYGRS |
| | | YYTMDYWGQGTTVTVSS |
| Lecanemab | 14 | DVVMTQSPLSLPVTPGAPASISCRSSQSI |
| light chain | | VHSNGNTYLEWYLQKPGQSPKLLIYKV |
| variable | | SNRFSGVPDRFSGSGSGTDFTLRISRVE |
| domain | | AEDVGIYYCFQGSHVPPTFGPGTKLEIK |
| E2814 heavy | 15 | EVQLLESGGGLVQPGGSLRLSCAASGY |
| chain | | TFTTYWITWVRQAPGKGLEWVSDIYPG |
| variable | | SSISNYNEKFKSRFTISVDNSKNTLYLQ |
| domain | | MNSLRAEDTAVYYCAREDGYDAWFA |
| | | YWGQGTLVTVSS |
| E2814 light | 16 | DIQMTQSPSSLSASVGDRVTITCRSSQSI |
| chain | | LHSNGNTYLEWYQQKPGKAPKLLISKV |
| variable | | SNRFSGVPSRFSGSGSGTDFTLTISSLQP |
| domain | | EDFATYYCFQGSHVPFTFGQGTKLEIK |

TABLE 13
Amino acid sequences of mAb constant regions

| mAb | IgG chain | Class | SEQ ID NO | Amino acid sequence |
|---|---|---|---|---|
| Lecanemab | Heavy chain | IgG1 | 17 | ASTKGPSVFPLAPSSKSTSGGT |
| | | | | AALGCLVKDYFPEPVTVSWN |
| | | | | SGALTSGVHTFPAVLQSSGLY |
| | | | | SLSSVVTVPSSSLGTQTYICNV |
| | | | | NHKPSNTKVDKRVEPKSCDK |
| | | | | THTCPPCPAPELLGGPSVFLFP |
| | | | | PKPKDTLMISRTPEVTCVVVD |
| | | | | VSHEDPEVKFNWYVDGVEVH |
| | | | | NAKTKPREEQYNSTYRVVSVL |
| | | | | TVLHQDWLNGKEYKCKVSNK |
| | | | | ALPAPIEKTISKAKGQPREPQV |
| | | | | YTLPPSREEMTKNQVSLTCLV |
| | | | | KGFYPSDIAVEWESNGQPENN |
| | | | | YKTTPPVLDSDGSFFLYSKLT |
| | | | | VDKSRWQQGNVFSCSVMHEA |
| | | | | LHNHYTQKSLSLSPGK |
| Lecanemab | Light chain | kappa | 18 | RTVAAPSVFIFPPSDEQLKSGT |
| | | | | ASVVCLLNNFYPREAKVQWK |
| | | | | VDNALQSGNSQESVTEQDSK |
| | | | | DSTYSLSSTLTLSKADYEKHK |
| | | | | VYACEVTHQGLSSPVTKSFNR |
| | | | | GEC |
| E2814 | Heavy chain | IgG1 | 19 | ASTKGPSVFPLAPSSKSTSGGT |
| | | | | AALGCLVKDYFPEPVTVSWN |
| | | | | SGALTSGVHTFPAVLQSSGLY |
| | | | | SLSSVVTVPSSSLGTQTYICNV |
| | | | | NHKPSNTKVDKKVEPKSCDK |
| | | | | THTCPPCPAPELLGGPSVFLFP |
| | | | | PKPKDTLMISRTPEVTCVVVD |
| | | | | VSHEDPEVKFNWYVDGVEVH |
| | | | | NAKTKPREEQYNSTYRVVSVL |
| | | | | TVLHQDWLNGKEYKCKVSNK |
| | | | | ALPAPIEKTISKAKGQPREPQV |
| | | | | YTLPPSRDELTKNQVSLTCLV |
| | | | | KGFYPSDIAVEWESNGQPENN |
| | | | | YKTTPPVLDSDGSFFLYSKLT |
| | | | | VDKSRWQQGNVFSCSVMHEA |
| | | | | LHNHYTQKSLSLSPGK |
| E2814 | Light chain | kappa | 20 | RTVAAPSVFIFPPSDEQLKSGT |
| | | | | ASVVCLLNNFYPREAKVQWK |
| | | | | VDNALQSGNSQESVTEQDSK |
| | | | | DSTYSLSSTLTLSKADYEKHK |
| | | | | VYACEVTHQGLSSPVTKSFNR |
| | | | | GEC |

TABLE 14
Amino acid sequences of biomarkers

| Bio- | SEQ | |
|---|---|---|
| marker | ID NO | Amino acid sequence |
| Aβ 1-40 | 21 | DAEFRHDSGYEVHHQKLVFFAEDVGSNKGAIIG |
| | | LMVGGVV |
| Aβ 1-42 | 22 | DAEFRHDSGYEVHHQKLVFFAEDVGSNKGAIIG |
| | | LMVGGVVIA |
| Tau | 23 | MAEPRQEFEVMEDHAGTYGLGDRKDQGGYTM |
| | | HQDQEGDTDAGLKESPLQTPTEDGSEEPGSETSD |
| | | AKSTPTAEDVTAPLVDEGAPGKQAAAQPHTEIP |
| | | EGTTAEEAGIGDTPSLEDEAAGHVTQARMVSKS |
| | | KDGTGSDDKKAKGADGKTKIATPRGAAPPGQK |
| | | GQANATRIPAKTPPAPKTPPSSGEPPKSGDRSGY |
| | | SSPGSPGTPGSRSRTPSLPTPPTREPKKVAVVRTP |
| | | PKSPSSAKSRLQTAPVPMPDLKNVKSKIGSTENL |
| | | KHQPGGGKVQIINKKLDLSNVQSKCGSKDNIKH |
| | | VPGGGSVQIVYKPVDLSKVTSKCGSLGNIHHKP |
| | | GGGQVEVKSEKLDFKDRVQSKIGSLDNITHVPG |
| | | GGNKKIETHKLTFRENAKAKTDHGAEIVYKSPV |
| | | VSGDTSPRHLSNVSSTGSIDMVDSPQLATLADEV |
| | | SASLAKQGL |

What is claimed is:

1) A method for treating or preventing Alzheimer's disease in a subject in need thereof, comprising administering to the subject:
(i) an isolated anti-Aβ protofibril antibody or antigen binding fragment thereof that is capable of binding to a human Aβ protofibril comprising
(a) a heavy chain variable domain comprising an amino acid sequence of SEQ ID NO:13, and
(b) a light chain variable domain comprising an amino acid sequence of SEQ ID NO:14, and
(ii) an anti-tau antibody or antigen binding fragment thereof that is capable of binding to human tau comprising
(a) a heavy chain variable domain comprising an amino acid sequence of SEQ ID NO:15, and
(b) a light chain variable domain comprising an amino acid sequence of SEQ ID NO:16,
wherein the anti-Aβ protofibril antibody or fragment thereof is administered in conjunction (e.g., simultaneously or sequentially) with the anti-tau antibody or fragment thereof.

2) The method of claim 1, wherein the isolated anti-Aβ protofibril antibody or fragment thereof comprises
(i) a heavy chain comprising an amino acid sequence of SEQ ID NO:17, and
(ii) a light chain comprising an amino acid sequence of SEQ ID NO:18.

3) The method of any one of claims 1 or 2, wherein the anti-tau antibody or fragment thereof comprises
(i) a heavy chain comprising an amino acid sequence of SEQ ID NO:19, and
(ii) a light chain comprising an amino acid sequence of SEQ ID NO:20.

4) The method of any one of claims 1-3, wherein the isolated anti-Aβ protofibril antibody or fragment thereof is administered once every week or one every two weeks.

5) The method of any one of claims 1-4, wherein the isolated anti-Aβ protofibril antibody or fragment thereof is administered intravenously, e.g., at a dose of 5 mg/kg-20 mg/kg (e.g., 10 mg/kg).

6) The method of any one of claims 1-5, wherein the isolated anti-Aβ protofibril antibody or fragment thereof is administered intravenously once every two weeks at a dose of 10 mg/kg, e.g., for at least 52 weeks.

7) The method of any one of claims 1-4, wherein the isolated anti-Aβ protofibril antibody or fragment thereof is administered subcutaneously, e.g., as a weekly administration comprising two consecutive subcutaneous injections, e.g., of 360 mg of the isolated anti-Aβ protofibril antibody or fragment thereof for a total dose of 720 mg, e.g., for at least 52 weeks.

8) The method of any one of claims 1-4 or 7, wherein the isolated anti-Aβ protofibril antibody or fragment thereof is administered subcutaneously for a period of time, e.g., at least 52 weeks or 18 months, and then a maintenance dose is administered.

9) The method of claim 8, wherein the maintenance dose comprises a subcutaneous administration of 720 mg weekly or biweekly.

10) The method of claim 8, wherein the maintenance dose comprises an intravenous administration of 10 mg/kg once every two weeks, once every four weeks, or once every twelve weeks.

11) The method of any one of claims 1-10, wherein the anti-tau antibody or fragment thereof is administered once every four weeks.

12) The method of any one of claims 1-11, wherein the anti-tau antibody or fragment thereof is administered in an amount of 1000-4500 mg (e.g., 1500, 3000, 4500 mg) once every four weeks.

13) The method of any one of claims 1-12, wherein the anti-tau antibody or fragment thereof is administered in an amount of 1500 mg once every four weeks.

14) The method of claim 1-12, wherein the anti-tau antibody or fragment thereof is administered in an amount of 3000 mg once every four weeks.

15) The method of any one of claims 1-11, wherein the anti-tau antibody or fragment thereof is administered at a dose of 3, 10, or 30 mg/kg once every four weeks.

16) The method of any one of claims 1-15, wherein the anti-tau antibody or fragment thereof is administered intravenously.

17) The method of any one of claims 1-16, wherein the anti-Aβ protofibril antibody or fragment thereof is administered before the start of treatment with the anti-tau antibody or fragment thereof.

18) The method of any one of claims 1-17, wherein the anti-Aβ protofibril antibody or fragment thereof is administered for at least 10 weeks (e.g., at least 15, 20, 24, or 25 weeks) before the start of treatment with the anti-tau antibody or fragment thereof.

19) The method of claim 17 or 18, wherein the subject is symptomatic for Alzheimer's disease.

20) The method of claim 19, wherein the subject has early-onset Alzheimer's disease.

21) The method of claim 16, wherein the anti-tau antibody or fragment thereof is administered by itself for 52 weeks before the start of treatment with the anti-tau antibody or fragment thereof in conjunction with the anti-Aβ protofibril antibody or fragment thereof.

22) The method of any one of claims 1-16, wherein the anti-tau antibody or fragment thereof is administered before the start of treatment with the anti-Aβ protofibril antibody or fragment thereof.

23) The method of any one of claims 1-16, wherein the anti-tau antibody or fragment thereof is administered for at least 25 weeks (e.g., at least 30, 40, 50, or 52 weeks) before administration of the anti-Aβ protofibril antibody or fragment thereof.

24) The method of claim 22 or 23, wherein the subject is asymptomatic for Alzheimer's disease.

25) The method of any one of claims 1-24, wherein the subject has a genetic mutation for dominantly inherited Alzheimer's disease, e.g., wherein the subject a genetic mutation in at least one of three genes—PSEN1, PSEN2, or APP.

26) The method of claim 25, wherein the subject has a mutation in APP.

27) The method of any one of claims 1-26, wherein the subject has a family history of Alzheimer's disease, e.g., a history of a family member being diagnosed with Alzheimer's disease before the age of 60.

28) The method of any of claims 1-27, wherein the tau spread, e.g., as measured by tau PET (such as MK-6240 Tau-PET) and/or MRI, is reduced after administration as compared to an untreated control subject.

29) The method of any of claims 1-28, wherein the level of phosphorylated tau 217 in a sample (e.g., a cerebrospinal fluid or blood sample) taken from the subject after administration is reduced relative to the level of phosphorylated tau 217 in a sample taken from the subject before administration.

30) The method of any of claims 1-29, wherein the Clinical Dementia Rating Scale Sum of Boxes score after administration is improved in the subject relative to a score in the subject prior to administration.

31) The method of any of claims 1-30, wherein the ADAS-cog (Alzheimer's disease Assessment Scale-cognitive subscale), MMSE (Mini-Mental State Evaluation), and/or Clinical Dementia Rating (CDR) score after administration is improved in the subject relative to a score prior to administration.

32) The method of any of claims 1-31, wherein the Aβ42/40 ratio in a sample (e.g., a blood sample, such as a plasma sample, or a CSF sample) taken from the subject after administration is increased relative to the ratio in a sample taken from the subject prior to administration.

33) The method of any of claims 1-32, wherein the level of Aβ protofibrils, e.g., as measured by PET (such as FDG-PET) and/or MRI, in the subject after administration is reduced as compared to the level of Aβ protofibrils in the subject prior to administration and/or as compared to an untreated control or subject.

34) A kit comprising
(i) an isolated anti-Aβ protofibril antibody or antigen binding fragment thereof that is capable of binding to human Aβ protofibrils comprising
(a) a heavy chain variable domain comprising an amino acid sequence of SEQ ID NO:13, and
(b) a light chain variable domain comprising an amino acid sequence of SEQ ID NO:14, and
(ii) an anti-tau antibody or antigen-binding fragment thereof that is capable of binding to human tau comprising
(a) a heavy chain variable domain comprising an amino acid sequence of SEQ ID NO:15, and
(b) a light chain variable domain comprising an amino acid sequence of SEQ ID NO:16.

35) The kit of claim 34, wherein the anti-Aβ protofibril antibody or fragment thereof is present in an amount suitable for intravenous administration at a dose of 10 mg/kg or suitable for subcutaneous administration of two consecutive 360 mg doses for a total dose of 720 mg, and the anti-tau antibody or fragment thereof is present in an amount suitable for administration at a dose of 1500 or 3000 mg.

36) The kit of any one of claim 34 or 35, wherein the anti-Aβ protofibril antibody or fragment thereof is present in an amount suitable for subcutaneous administration of two consecutive 360 mg doses for a total dose of 720 mg, and the anti-tau antibody or fragment thereof is present in an amount suitable for administration at a dose of 3000 mg.

37) The kit of any one of claims 34-36, wherein the anti-Aβ protofibril antibody or fragment thereof and the anti-tau antibody or antigen-binding fragment thereof are formulated in separate containers within the kit.

38) The kit of any one of claims 34-37, wherein the isolated anti-Aβ protofibril antibody or fragment thereof comprises
(i) a heavy chain comprising an amino acid sequence of SEQ ID NO:17, and
(ii) a light chain comprising an amino acid sequence of SEQ ID NO:18.

39) The kit of any one of claims 34-38, wherein the anti-tau antibody or fragment thereof comprises
(i) a heavy chain comprising an amino acid sequence of SEQ ID NO:19, and
(ii) a light chain comprising an amino acid sequence of SEQ ID NO:20.

40) A pharmaceutical combination comprising:
(i) an isolated anti-Aβ protofibril antibody or antigen binding fragment thereof that is capable of binding to human Aβ protofibrils comprising
(a) a heavy chain variable domain comprising an amino acid sequence of SEQ ID NO:13, and
(b) a light chain variable domain comprising an amino acid sequence of SEQ ID NO:14, and
(ii) an anti-tau antibody or antigen-binding fragment thereof that is capable of binding to human tau comprising
(a) a heavy chain variable domain comprising an amino acid sequence of SEQ ID NO:15, and
(b) a light chain variable domain comprising an amino acid sequence of SEQ ID NO:16.

41) The pharmaceutical combination of claim 40, wherein the anti-Aβ protofibril antibody or fragment thereof is present in an amount suitable for intravenous administration at a dose of 10 mg/kg or suitable for subcutaneous administration at a dose of 720 mg and the anti-tau antibody or fragment thereof is present in an amount suitable for administration at a dose of 1500 or 3000 mg.

42) Use of an anti-Aβ protofibril antibody or fragment thereof and an anti-tau antibody or fragment thereof in treating or preventing Alzheimer's disease, wherein:
(i) an isolated anti-Aβ protofibril antibody or antigen binding fragment thereof that is capable of binding to human Aβ protofibrils comprising
(a) a heavy chain variable domain comprising an amino acid sequence of SEQ ID NO:13, and
(b) a light chain variable domain comprising an amino acid sequence of SEQ ID NO:14, and
(ii) an anti-tau antibody or antigen-binding fragment thereof that is capable of binding to human tau comprising
(a) a heavy chain variable domain comprising an amino acid sequence of SEQ ID NO:15, and
(b) a light chain variable domain comprising an amino acid sequence of SEQ ID NO:16.

43) The use of claim 42, wherein the anti-AP protofibril antibody or fragment thereof is present in an amount suitable for intravenous administration at a dose of 10 mg/kg or suitable for subcutaneous administration at a dose of 720 mg and the anti-tau antibody or fragment thereof is present in an amount suitable for intravenous administration at a dose of 1500 or 3000 mg.

44) The use of any one of claim 42 or 43, for use in treating or preventing early-onset Alzheimer's disease in a subject.

45) The use of any one of claims 42-44, wherein the isolated anti-Aβ protofibril antibody or fragment thereof is administered once every week or once every two weeks.

46) The use of any one of claims 42-45, wherein the isolated anti-Aβ protofibril antibody or fragment thereof is administered intravenously at a dose of 10 mg/kg.

47) The use of any one of claims 42-45, wherein the isolated anti-Aβ protofibril antibody or fragment thereof is administered subcutaneously in two consecutive 360 mg doses for a total dose of 720 mg.

48) The use of any one of claims 42-47, wherein the anti-tau antibody or fragment thereof is administered once every four weeks.

49) The use of any one of claims 42-48, wherein the anti-tau antibody or fragment thereof is administered intravenously at a dose of 1500 or 3000 mg once every four weeks.

50) The use of any one of claims 42-49, wherein the anti-tau antibody or fragment thereof is administered intravenously at a dose of 3000 mg once every four weeks.

51) The use of any one of claims 42-48, wherein the anti-tau antibody or fragment thereof is administered intravenously at a dose of 3, 10, or 30 mg/kg once every four weeks.

52) The use of any one of claims 42-51, wherein the anti-Aβ protofibril antibody or fragment thereof is administered before the start of treatment with the anti-tau antibody or fragment thereof.

53) The use of any one of claims 42-52, wherein the anti-Aβ protofibril antibody or fragment thereof is administered for at least 10 weeks (e.g., at least 15, 20, 24, or 25 weeks) before the start of treatment with the anti-tau antibody or fragment thereof.

54) The use of any one of claims 42-53, wherein the anti-tau antibody or fragment thereof is administered by itself for 52 weeks before the start of treatment with the anti-tau antibody or fragment thereof in conjunction with the anti-Aβ protofibril antibody or fragment thereof.

55) The use of any one of claims 42-51, wherein the anti-tau antibody or fragment thereof is administered before the start of treatment with the anti-Aβ protofibril antibody or fragment thereof.

56) The use of any one of claims 42-51 or 55, wherein the anti-tau antibody or fragment thereof is administered for at least 25 weeks (e.g., at least 30, 40, 50, or 52 weeks) before administration of the anti-Aβ protofibril antibody or fragment thereof.

57) The use of any one of claims 42-56, wherein the subject has a genetic mutation for dominantly inherited Alzheimer's disease, e.g., wherein the subject a genetic mutation in at least one of three genes—PSEN1, PSEN2, or APP.

58) The use of any one of claims 42-57, wherein the subject has a family history of Alzheimer's disease, e.g., a history of a family member being diagnosed with Alzheimer's disease before the age of 60.

59) The use of any one of claims 42-58, wherein the tau spread, e.g., as measured by tau PET (such as MK-6240 Tau-PET) and/or MRI, is reduced after administration as compared to an untreated control subject.

60) The use of any one of claims 42-59, wherein the level of phosphorylated tau 217 in a sample (e.g., a cerebrospinal fluid or blood sample) taken from the subject after administration is reduced relative to the level of phosphorylated tau 217 in a sample taken from the subject before administration.

61) The use of any one of claims 42-60, wherein the Clinical Dementia Rating Scale Sum of Boxes score after administration is improved in the subject relative to a score in the subject prior to administration.

62) The use of any one of claims 42-61, wherein the ADAS-cog (Alzheimer's disease Assessment Scale-cognitive subscale), MMSE (Mini-Mental State Evaluation), and/or Clinical Dementia Rating (CDR) score after administration is improved in the subject relative to a score prior to administration.

63) The use of any one of claims 42-62, wherein the Aβ42/40 ratio in a sample (e.g., a blood sample, such as a plasma sample, or a CSF sample) taken from the subject after administration is increased relative to the ratio in a sample taken from the subject prior to administration.

64) The use of any one of claims 42-63, wherein the level of Aβ protofibrils, e.g., as measured by PET (such as FDG-PET) and/or MRI, in the subject after administration is reduced as compared to the level of Aβ protofibrils in the subject prior to administration and/or as compared to an untreated control or subject.

65) The use of any one of claims 42-64, wherein the isolated anti-Aβ protofibril antibody or fragment thereof comprises
(i) a heavy chain comprising an amino acid sequence of SEQ ID NO:17, and
(ii) a light chain comprising an amino acid sequence of SEQ ID NO:18.

66) The use of any one of claims 42-65, wherein the anti-tau antibody or fragment thereof comprises
(i) a heavy chain comprising an amino acid sequence of SEQ ID NO:19, and
(ii) a light chain comprising an amino acid sequence of SEQ ID NO:20.
